(19) United States
(12) Patent Application Publication
(10) Pub. No.: US 2025/0352453 A1
(43) Pub. Date: Nov. 20, 2025

(54) COMPOSITION COMPRISING A POLYHYDROXYALKANOATE COPOLYMER WITH A LONG HYDROCARBON-BASED CHAIN BEARING IONIC GROUP(S), AND METHOD FOR TREATING KERATIN MATERIALS USING THE COMPOSITION

(71) Applicant: L'Oreal, Paris (FR)

(72) Inventors: Julien PORTAL, Aulnay-sous-Bois (FR); Etienne SAVONNET, Aulnay-sous-Bois (FR); Romain GARCON, Aulnay-sous-Bois (FR)

(73) Assignee: L'Oreal, Paris (FR)

(21) Appl. No.: 18/716,673

(22) PCT Filed: Dec. 20, 2022
(86) PCT No.: PCT/EP2022/087122

§ 371 Date: Jun. 5, 2024

(30) Foreign Application Priority Data
Dec. 20, 2021 (FR) 2114049

(51) Int. Cl. A61K 8/85; A61Q 5/00; A61Q 19/00

(52) U.S. Cl. CPC A61K 8/85; A61Q 5/00; A61Q 19/00

(57) ABSTRACT

The present invention relates to a method for treating keratin materials, preferably α) keratin fibres, notably human keratin fibres such as the hair, or β) human skin, in particular the lips, using a composition, preferably a cosmetic composition, comprising a) one or more polyhydroxyalkanoate (PHA) copolymers a) comprising at least two different repeating polymer units chosen from the following units (A) and (B), and also the optical or geometric isomers thereof, the organic or mineral acid or base salts thereof, and the solvates thereof such as hydrates: —[—O—CH(R¹)—CH²—C(O)—]— unit (A) —[—O—CH(R²)—CH²—C(O)—]— unit (B) in which polymer units (A) and (B): —R¹ and R² are as defined in the description; and it being understood that: —(A) is different from (B); —the composition comprises an amount of less than or equal to 2% by weight of surfactants, relative to the total weight of the composition.

The present invention relates to a composition, preferably a cosmetic composition, comprising at least one polyhydroxyalkanoate (PHA) copolymer with a hydrocarbon-based chain bearing ionic group(s), particular ionic PHAs, the use of the composition in cosmetics and also a method for treating keratin materials using the composition.

It is known practice to use, in cosmetics, film-forming polymers which can be conveyed in organic media, such as hydrocarbon-based oils. Polymers are notably used as film-forming agents in makeup products such as mascaras, eyeliners, eyeshadows or lipsticks.

FR-A-2964663 describes a cosmetic composition comprising pigments coated with a C₃-C₂₁ polyhydroxyalkanoate, such as poly(hydroxybutyrate-co-hydroxyvalerate).

WO 2011/154508 describes a cosmetic composition comprising a 4-carboxy-2-pyrrolidinone ester derivative and a film-forming polymer which may be a polyhydroxyalkanoate, such as polyhydroxybutyrate, polyhydroxyvalerate and polyhydroxybutyrate-co-polyhydroxyvalerate.

US-A-20151274972 describes a cosmetic composition comprising a thermoplastic resin, such as a polyhydroxyalkanoate, in aqueous dispersion and a silicone elastomer.

The majority of the polyhydroxyalkanoates are polymers derived from the polycondensation of polymeric repeating units that are for the most part identical and derived from the same carbon source or substrate. These documents do not describe the use of a copolymer derived from polycondensation using an aliphatic substrate or first carbon source, and at least one second substrate different from the first, comprising one or more ionic groups, of different chemical nature from the first carbon source.

There is therefore a need to provide a composition comprising PHAs with diverse functionalization or which are functionalizable and which can subsequently become more water-soluble despite the presence of a long aliphatic chain. This would make it possible to obtain a film on keratin materials which has good cosmetic properties, notably good resistance to oils and to sebum, and also to be able to modify the sheen or the mattness.

Consumers are increasingly seeking cosmetic formulae with a low content of surfactants (<2% by weight relative to the total weight of the formula), or even without any surfactants. However it is known that it is difficult with a long-chain PHAs of mcl-PHA type, notably that are carboxylated, to have stable particles without having to use a surfactants [cf, “Stability of Aqueous Suspensions of Medium-Chain-Length Poly-3-Hydroxyalkanoate Particles”, Journal of Polymers and the Environment, vol. 24, p. 281-285 (2016)].

Unexpectedly, it has been discovered that polyhydroxyalkanoate copolymers with hydrocarbon-based chains bearing ionic group(s), as defined below, may be easily used in aqueous media (particularly with predominantly water as solvent), thus making it possible to obtain easy-to use homogeneous compositions, even when using very little, or even no, surfactant.

In addition, the composition has good stability, notably after storage for one month at room temperature (25° C.). The composition, notably after its application to keratin materials, makes it possible to obtain a film having good cosmetic properties, in particular good resistance to oils and to sebum, and also a matt or shiny appearance. Furthermore, via the ionic groups, it is possible to make the PHA polymers interact with active agents, notably organic active agents, such as UV screening agents, fluorescent or non-fluorescent chromophores, anti-ageing active agents, said agents comprising ionic groups of opposite ionicity to the PHA polymers and it being possible for said active agents to then become more persistent once grafted, notably with respect to oils, water and sebum.

Thus, the first subject of the present invention is a method for treating keratin materials, preferably α) keratin fibres, notably human keratin fibres such as the hair, or β) human skin, in particular the lips, which uses at least one composition, preferably a cosmetic composition, comprising a) one or more PHA copolymers a) comprising at least two different repeating polymer units chosen from the following units (A) and (B), and also the optical or geometric isomers thereof, the organic or mineral acid or base salts thereof, and the solvates thereof such as hydrates:

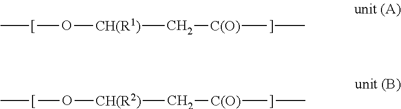

in which polymer units (A) and (B):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms; preferably, the hydrocarbon-based chain is chosen from i) linear or branched (C₅-C₂₈)alkyl, ii) linear or branched (C₆-C₂₈)alkenyl, iii) linear or branched (C₆-C₂₈)alkynyl, iii) preferably the hydrocarbon-based chain is non-cyclic and linear;
said hydrocarbon-based chain being:
   substituted with one or more groups chosen from anionic groups (A), cationic groups (C′) and mixtures thereof;
      the A⁻ group(s) being chosen from —C(O)OH, —C(O)O, —SOH, —S(O)₂O⁻, —O—S(O)—OH, —O—S(O)₂O⁻, —P(O)OH₂, —P(O)O₂, —P(O)₂O⁻, —P(OH)₂, ═P(O)—OH, —P(OH)O⁻, ═P(O)—O⁻, ═P—OH, and ═P—O⁻ and mixtures thereof, the anionic parts comprise one or more cationic counterions M⁺ to achieve the electroneutrality of the PHA such as i) an alkali metal, ii) an alkaline-earth metal, iii) ammonium RₐR_{b}R_{c}R_{d}N⁺, iv) phosphonium RₐR_{b}R_{c}R_{d}P⁺ with Rₐ as defined below v) the cationic forms of lysine or guanidine;
      the C⁺ cationic group(s) being chosen from cationizable amine groups —NR_{b}R_{c}, ammonium groups —N⁺RₐR_{b}R_{c}, phosphonium groups —P⁺RₐR_{b}R_{c}, cationizable amidine groups —C(═NRₐ)—NR_{b}R_{c}, amidinium groups —C(═NRₐ)—N⁺R_{b}R_{c}R_{d}, cationizable guanidine groups —N(Rₐ)—C(═NRₐ)—NR_{b}R_{c}, guanidinium groups —N(Rₐ)—C(═NRₐ)—N⁺R_{b}R_{c}R_{d}, or cationizable imidazolyl groups optionally substituted with one or more Rₐ radicals as defined below, or imidazolium groups optionally substituted with one or more Rₐ radicals as defined below, and mixtures thereof, the cationic parts comprising one or more anionic counterions
      Q⁻ to achieve the electroneutrality of the PHA, such as a halide, a carboxylate such as acetate or glutamate, with
      Rₐ representing a hydrogen atom, a (C₁-C₄)alkyl group, or (hetero)aryl(C₁-C₄)alkyl group such as benzyl, preferably Rₐ represents a hydrogen atom or a (C₁-C₄)alkyl group; R_{b}, R_{c}, and R_{d}, which may be identical or different, represent a (C₁-C₄)alkyl group optionally substituted with one or more hydroxy, (hetero)aryl, (hetero)aryl(C₁-C₄)alkyl such as benzyl, (hetero)cycloalkyl or (hetero)cycloalkyl(C₁-C₄)alkyl groups, or else R_{b} and R_{c}, or R_{c} and R_{d} represent, with the nitrogen or phosphorus atom which bears them, a saturated or unsaturated, aromatic or non-aromatic heterocycle; particularly a (C₁-C₄)alkyl optionally substituted with one or more hydroxy groups; the cationic parts comprising one or more anionic counterions Q⁻ to ensure electroneutrality;
      it being possible for the R¹ radicals to also be substituted with one or more atoms or groups chosen from: a) halogen such as chlorine or bromine, b) hydroxy, c) thiol, d) (di)(C₁-C₄)(alkyl)amino, e) thiocarboxy, f) (thio)carboxamide —C(O)—N(Rₐ)₂ or —C(S)—N(Rₐ)₂, g) cyano, h) iso(thio)cyanate, i) (hetero)aryl such as phenyl or furyl, and j) (hetero)cycloalkyl, k) cosmetic active agent; l) R-X with R representing a group chosen from α) cycloalkyl such as cyclohexyl, β) heterocycloalkyl such as sugar, preferably monosaccharide such as glucose, γ) (hetero)aryl such as phenyl, m) thiosulfate; X representing a′) O, S, N(R′ₐ) or Si(R′_{b})(R′_{c}), b′) S(O)ᵣ, or (thio)carbonyl. c′) or combinations of a′) with b′) such as (thio)ester, (thio)amide, (thio)urea or sulfonamide; R′ₐ representing a hydrogen atom, or a (C₁-C₄)alkyl group, or aryl(C₁-C₄)alkyl group such as benzyl, preferably Rₐ represents a hydrogen atom; R′_{b} and R′_{c}, which may be identical or different, represent a (C₁-C₄)alkyl or (C₁-C₄)alkoxy group, particularly only one substituent, preferably chosen from α) halogen, and j) such as epoxide and
optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide, preferably ester —O—C(O)— or —C(O)—O— with r being equal to 1 or 2, Rₐ being as defined previously; preferably, Rₐ represents a hydrogen atom, R_{b} and R_{c} being as defined previously;
   R² represents a hydrocarbon-based chain as defined for R¹; preferably the hydrocarbon-based chain of the radical R² has a carbon number corresponding to the number of carbon atoms in the radical R¹ minus at least one carbon atom, preferably corresponding to the number of carbon atoms in the radical R minus two carbon atoms;
b) optionally water;
it being understood that:
   the R¹ radicals may comprise, in the same PHA, A⁻ and C⁺ groups in which case the counterions M⁺ and Q⁻ may be absent while ensuring the electroneutrality of the PHA
   (A) is different from (B);
   the R¹ and R² radicals may comprise, in the same PHA, A⁻ and C⁺ groups in which case the counterions M⁺ and Q⁻ may be absent while ensuring the electroneutrality of the PHA;
   the composition comprises an amount of less than or equal to 2% by weight of surfactants, relative to the total weight of the composition, particularly less than or equal to 1% by weight of surfactants, more particularly less than 0.5% by weight of surfactants, relative to the total weight of the composition, preferentially the composition is free of surfactant.

According to a variant, a method for treating keratin materials, preferably α) keratin fibres, notably human keratin fibres such as the hair, or β) human skin, in particular the lips, can be a method which uses at least one composition, preferably a cosmetic composition, comprising a) one or more PHA copolymers a) comprising one ore more following units (A), and also the optical or geometric isomers thereof, the organic or mineral acid or base salts thereof, and the solvates thereof such as hydrates:

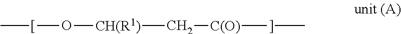

in which polymer units (A):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms; preferably, the hydrocarbon-based chain is chosen from i) linear or branched (C₅-C₂₈)alkyl, ii) linear or branched (C₆-C₂₈)alkenyl, iii) linear or branched (C₆-C₂₈)alkynyl, iii) preferably the hydrocarbon-based chain is non-cyclic and linear;
said hydrocarbon-based chain being:
   substituted with one or more groups chosen from anionic groups (A⁻), cationic groups (C⁺) and mixtures thereof;
      the A⁻ group(s) being chosen from —C(O)OH, —C(O)O⁻, —SO₃H, —S(O)₂O⁻, —O—S(O)₂OH, —O—S(O)₂O⁻, —P(O)OH₂, —P(O)O₂⁻, —P(O)₂O⁻, —P(OH)₂, ═P(O)—OH, —P(OH)O⁻, ═P(O)—O⁻, ═P—OH, and ═P—O⁻ and mixtures thereof, the anionic parts comprise one or more cationic counterions M⁺ to achieve the electroneutrality of the PHA such as i) an alkali metal, ii) an alkaline-earth metal, iii) ammonium RₐR_{b}R_{c}R_{d}N⁺, iv) phosphonium RₐR_{b}R_{c}R_{d}P⁺ with Rₐ as defined below v) the cationic forms of lysine or guanidine;
      the C⁺ cationic group(s) being chosen from cationizable amine groups —NR_{b}R_{c}, ammonium groups —N⁺RₐR_{b}R_{c}, phosphonium groups —P⁺RₐR_{b}R_{c}, cationizable amidine groups —C(═NRₐ)—NR_{b}R_{c}, amidinium groups —C(═NRₐ)—N⁺R_{b}R_{c}R_{d}, cationizable guanidine groups —N(Rₐ)—C(═NRₐ)—NR_{b}R_{c}, guanidinium groups —N(Rₐ)—C(═NRₐ)—N⁺R_{b}R_{c}R_{d}, or cationizable imidazolyl groups optionally substituted with one or more Rₐ radicals as defined below, or imidazolium groups optionally substituted with one or more Rₐ radicals as defined below, and mixtures thereof, the cationic parts comprising one or more anionic counterions Q⁻ to achieve the electroneutrality of the PHA, such as a halide, a carboxylate such as acetate or glutamate; with
      Rₐ representing a hydrogen atom, a (C₁-C₄)alkyl group, or (hetero)aryl(C₁-C₄)alkyl group such as benzyl, preferably Rₐ represents a hydrogen atom or a (C₁-C₄)alkyl group; R_{b}, R_{c}, and R_{d}, which may be identical or different, represent a (C₁-C₄)alkyl group optionally substituted with one or more hydroxy, (hetero)aryl, (hetero)aryl(C₁-C₄)alkyl such as benzyl, (hetero)cycloalkyl or (hetero)cycloalkyl(C₁-C₄)alkyl groups, or else R_{b} and R_{c}, or R_{c} and R_{d} represent, with the nitrogen or phosphorus atom which bears them, a saturated or unsaturated, aromatic or non-aromatic heterocycle; particularly a (C₁-C₄)alkyl optionally substituted with one or more hydroxy groups; the cationic parts comprising one or more anionic counterions Q⁻ to ensure electroneutrality;
      it being possible for the R¹ radicals to also be substituted with one or more atoms or groups chosen from: a) halogen such as chlorine or bromine, b) hydroxy, c) thiol, d) (di)(C₁-C₄)(alkyl)amino, e) thiocarboxy, f) (thio)carboxamide —C(O)—N(Rₐ)₂ or —C(S)—N(Rₐ)₂, g) cyano, h) iso(thio)cyanate, i) (hetero)aryl such as phenyl or furyl, and j) (hetero)cycloalkyl, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, m) thiosulfate; X representing a′) O, S, N(R′ₐ) or Si(R′_{b})(R′_{c}), b′) S(O)ᵣ, or (thio)carbonyl, c′) or combinations of a′) with b′) such as (thio)ester, (thio)amide, (thio)urea or sulfonamide; R′ₐ representing a hydrogen atom, or a (C₁-C₄)alkyl group, or aryl(C₁-C₄)alkyl group such as benzyl, preferably Rₐ represents a hydrogen atom; R′_{b} and R′_{c}, which may be identical or different, represent a (C₁-C₄)alkyl or (C₁-C₄)alkoxy group, particularly only one substituent, preferably chosen from a) halogen, and j) such as epoxide and optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide, preferably ester —O—C(O)— or —C(O)—O— with r being equal to 1 or 2, Rₐ being as defined previously; preferably, Rₐ represents a hydrogen atom, R_{b} and R_{c} being as defined previously;
b) optionally water;
it being understood that:
   the R¹ radicals may comprise, in the same PHA, A⁻ and C⁺ groups in which case the counterions M⁺ and Q⁻ may be absent while ensuring the electroneutrality of the PHA;
the composition comprises an amount of less than or equal to 2% by weight of surfactants, relative to the total weight of the composition, particularly less than or equal to 1% by weight of surfactants, more particularly less than 0.5% by weight of surfactants, relative to the total weight of the composition, preferentially the composition is free of surfactant

In one variant, a composition, preferably a cosmetic composition, as defined previously and hereinbelow, can comprise a) one or more polyhydroxyalkanoate (PHA) copolymers, containing one or more units (A), wherein when R¹ represents a hydrocarbon-based chain which is saturated and linear comprising 3 to 10 carbon atoms and substituted with one or more carboxyl —C(O)OH groups then said chain is interrupted with heteroatoms or groups a′) to c′) as defined previously.

In another subject a PHA copolymer can be a PHA copolymer as defined previously in which (A) is such that:
R¹ represents a saturated or unsaturated, linear or branched, non-cyclic hydrocarbon-based chain, or a saturated or unsaturated non-aromatic cyclic hydrocarbon-based chain, comprising from 5 to 28 carbon atoms; preferably, the hydrocarbon-based chain is chosen from i) linear or branched (C₅-C₂₈)alkyl, ii) linear or branched (C₆-C₂₈)alkenyl, iii) linear or branched (C₆-C₂₈)alkynyl, iii) preferably the hydrocarbon-based chain is non-cyclic and linear; said hydrocarbon-based chain being:
substituted with one or more anionic groups (A⁻), cationic groups (C⁺) and mixtures thereof;
   the A⁻ group(s) being chosen from —C(O)O⁻, —SO₃H, —S(O)₂O⁻, —O—S(O)₂OH, —O—S(O)₂O⁻, —P(O)OH₂, —P(O)O₂, —P(O)₂O⁻, —P(OH)₂, ═P(O)—OH, —P(OH)O⁻, ═P(O)—O⁻, ═P—OH, and ═P—O⁻ and mixtures thereof, the anionic parts comprise one or more cationic counterions M⁺;
   the C⁺ cationic group(s) being chosen from cationizable amine groups —NR_{b}R_{c}, ammonium groups —N⁺RₐR_{b}R_{c}, phosphonium groups —P*RₐR_{b}R_{c}, cationizable amidine groups —C(═NRₐ)—NR_{b}R_{c}, amidinium groups —C(═NRₐ)—N⁺R_{b}R_{c}R_{d}, cationizable guanidine groups —N(Rₐ)—C(═NRₐ)—NR_{b}R_{c}, guanidinium groups —N(Rₐ)—C(═NRₐ)—N⁺R_{b}R_{c}R_{d}, or cationizable imidazolyl groups optionally substituted with one or more Rₐ radicals as defined previously, or imidazolium groups optionally substituted with one or more Rₐ radicals as defined previously, and mixtures thereof to achieve the electroneutrality of the PHA, with Rₐ, R_{b}, R_{c}, and R_{d} as defined previously, the cationic parts comprising one or more anionic counterions Q⁻, M⁺ and Q⁻ being as defined previously;
In one variant, the PHA copolymer as defined previously in which (A) is such that R¹ represents a hydrocarbon-based chain, notably i) linear or branched (C₅-C₂₅)alkyl, which is substituted with one or more (preferably one) A⁻ groups chosen from —C(O)O⁻ M⁺ with M⁺ as above, preferably ammonium RₐR_{b}R_{c}R_{d}N⁺, with Rₐ, R_{b}, R_{c} and R_{d} as defined previously, preferably Rₐ represents a hydrogen atom; R_{b} and R_{c}, which may be identical or different, preferably identical, represent a linear or branched, preferably branched (C₁-C₄)alkyl group such as isopropyl or isobutyl, more preferentially isopropyl; and R_{d} represents a linear or branched (C₁-C₄)alkyl group, such as ethyl, more particularly M⁺ represents a diisopropylethylammonium group: (i-Pr)₂N⁺(H)Et.

In one variant, a process can be a process for preparing PHA bearing a carboxylate group.

In one variant, a non-therapeutic cosmetic method for treating keratin materials can comprise the application to the keratin materials of the composition, or of the PHA copolymer, as defined previously or as defined hereinbelow. The treatment method is in particular a method for caring for or making up keratin materials.

In one variant, a non-therapeutic use, can be a cosmetic use of the composition, or of the PHA copolymer, as defined previously or as defined hereinbelow, notably for caring for or making up keratin materials.

For the purposes of the present invention and unless otherwise indicated:
the term “cosmetic active agent” means the radical of an organic or organosilicon compound which can be integrated into a cosmetic composition to give an effect on keratin materials, whether this effect is immediate or provided by repeated applications. As examples of cosmetic active agents, mention may be made of coloured or colourless, fluorescent or non-fluorescent chromophores such as those derived from optical brighteners, or chromophores derived from UVA and/or UVB screening agents, anti-ageing active agents or active agents intended for providing a benefit to the skin such as active agents having action on the barrier function, deodorant active agents other than mineral particles, antiperspirant active agents other than mineral particles, desquamating active agents, antioxidant active agents, moisturizing active agents, sebum-regulating active agents, active agents intended for limiting the sheen of the skin, active agents intended for combating the effects of pollution, antimicrobial or bactericidal active agents, antidandruff active agents, and fragrances.
the term “(hetero)aryl” means aryl or heteroaryl groups;
the term “(hetero)cycloalkyl” means cycloalkyl or heterocycloalkyl groups;
the “aryl” or “heteroaryl” radicals or the aryl or heteroaryl part of a radical may be substituted with at least one substituent borne by a carbon atom, chosen from:
   a C₁-C₆ and preferably C₁-C₄-alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆ and preferably C₁-C₄alkyl radicals;
   an acylamino radical (—NR—COR′) in which the R radical is a hydrogen atom;
   a C₁-C₄ alkyl radical and the R′ radical is a C₁-C₄ alkyl radical; a carbamoyl radical ((R)₂N—CO—) in which the R radicals, which may be identical or different, represent a hydrogen atom or a C₁-C₄ alkyl radical;
   an alkylsulfonylamino radical (R′SO—NR—) in which the R radical represents a hydrogen atom or a C₁-C₄ alkyl radical and the R′ radical represents a C₁-C₄ alkyl radical, or a phenyl radical;
   an aminosulfonyl radical ((R)₂N—S(O)₂—) in which the R radicals, which may be identical or different, represent a hydrogen atom or a C₁-C₄ alkyl radical;
   a carboxyl radical in the acid or salified form (preferably salified with an alkali metal or a substituted or unsubstituted ammonium);
   a cyano group (CN);
   a polyhalo(C₁-C₄)alkyl group, preferentially trifluoromethyl (CFs);
the cyclic or heterocyclic part of a non-aromatic radical may be substituted with at least one substituent borne by a carbon atom, chosen from the groups:
   hydroxyl,
   C₁-C₄ alkoxy, C₂-C₄ (poly)hydroxyalkoxy,
   alkylcarbonylamino (RCO—NR′—), in which the R″ radical is a hydrogen atom or a C₁-C₄ alkyl radical and the R radical is a C₁-C₂ alkyl radical or an amino radical substituted with one or two identical or different C₁-C₄ alkyl groups;
   alkylcarbonyloxy (RCO—O—), in which the R radical is a C₁-C₄ alkyl radical or an amino radical substituted with one or two identical or different C₁-C₄ alkyl groups;
   alkoxycarbonyl ((RO—CO—) in which the R radical is a C₁-C₄ alkyl radical or an amino radical substituted with one or two identical or different C₁-C₄ alkyl groups;
a cyclic or heterocyclic radical, or a non-aromatic part of an aryl or heteroaryl radical, may also be substituted with one or more oxo groups;
a hydrocarbon-based chain is unsaturated when it includes one or more double bonds and/or one or more triple bonds;
an “aryl” radical represents a monocyclic or fused or non-fused polycyclic group containing from 6 to 22 carbon atoms, and at least one ring of which is aromatic; preferentially, the aryl radical is a phenyl, biphenyl, naphthyl, indenyl, anthracenyl or tetrahydronaphthyl, preferably phenyl;
a “heteroaryl” radical represents a monocyclic or fused or non-fused polycyclic, 5- to 22-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl;
a “cyclic” or “cycloalkyl” radical is a monocyclic or fused or non-fused polycyclic, non-aromatic cyclic hydrocarbon-based radical containing from 5 to 22 carbon atoms, which may include one or more unsaturations; the cycloalkyl is preferably a cyclohexyl group;
a “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl and morpholinyl;
an “alkyl” radical is a linear or branched, in particular C₁-C₆ and preferably C₁-C₄ saturated hydrocarbon-based radical;
an “alkenyl” radical is a linear or branched unsaturated hydrocarbon-based radical comprising one or more conjugated or non-conjugated double bonds;
an “alkynyl” radical is a linear or branched unsaturated hydrocarbon-based radical comprising one or more conjugated or non-conjugated triple bonds;
an “alkoxy radical” is an alkyl-oxy radical for which the alkyl radical is a linear or branched C₁-C₆ and preferentially C₁-C₄ hydrocarbon-based radical;
the term “organic or mineral acid salt” more particularly means organic or mineral acid salts in particular chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) alkoxysulfinic acids: Alk-O—S(O)OH such as methoxysulfinic acid and ethoxysulfinic acid; vii) aryloxysulfinic acids such as tolueneoxysulfinic acid and phenoxysulfinic acid; viii) phosphoric acid H₃PO₄; ix) triflic acid CF₃SO₃H and x) tetrafluoroboric acid HBF₄; xi) organic carboxylic acids R^{∘}—C(O)—OH (I′), in which formula (I′) R^{∘} represents a (hetero)aryl group such as phenyl, (hetero)aryl(C₁-C₄)alkyl group such as benzyl, or (C₁-C₁₀)alkyl, said alkyl group being optionally substituted preferably with one or more hydroxyl groups or amino or carboxyl radicals, R^{∘} preferably denoting a (C₁-C₆)alkyl group optionally substituted with 1, 2 or 3 hydroxyl or carboxyl groups; more preferentially, the monocarboxylic acids of formula (I′) are chosen from acetic acid, glycolic acid, lactic acid, and mixtures thereof, and more particularly from acetic acid and lactic acid; and the polycarboxylic acids are chosen from tartaric acid, succinic acid, fumaric acid, citric acid and mixtures thereof; and xii) amino acids including more carboxylic acid radicals than amino groups, such as γ-carboxyglutamic acid, aspartic acid or glutamic acid, in particular γ-carboxyglutamic acid;
an “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)O⁻ such as benzenesulfonate and toluenesulfonate or tosylate; v) carboxylates notably of formula R′[C(O)OH]ᵤ with R′ representing a saturated or unsaturated, linear or branched hydrocarbon-based chain comprising from 1 to 8, notably from 1 to 6, preferably from 1 to 4 carbon atoms, which is optionally substituted by one or more hydroxyl or (di)(C₁-C₆)(alkyl)amino groups and u representing an integer between 1 and 6, better still between 1 and 4, even better still 1, 2 or 3 such as citrate; succinate; tartrate; lactate; acetate, or glutamate, vi) alkylsulfates: Alk-O—S(O)O⁻ such as methyl sulfate and ethyl sulfate; vii) aryl sulfates: Ar—O—S(O)O⁻ such as benzene sulfate and toluene sulfate; viii) alkoxy sulfates: Alk-O—S(O)₂O⁻ such as methoxy sulfate and ethoxy sulfate; ix) aryloxy sulfates: Ar—O—S(O)₂O⁻, x) phosphate; xi) triflate; and xii) borates such as tetrafluoroborate;
a “cationic counterion” is a cation or a cationic group associated with the anionic charge; more particularly the cationic counterion is chosen from i) alkali metals such as sodium or potassium, preferably Na⁺; ii) alkaline-earth metals such as Ca²⁺, Mg²⁺, iii) ammonium RₐR_{b}R_{c}R_{d}N⁺, iv) phosphonium RₐR_{b}R_{c}R_{d}P⁺ with Rₐ representing a hydrogen atom, or a (C₁-C₄)alkyl group or (hetero)aryl(C₁-C₄)alkyl group such as benzyl, preferably Rₐ represents a hydrogen atom or a (C₁-C₄)alkyl group; R_{b}, R_{c}, and R_{d}, which may be identical or different, represent a (C₁-C₄)alkyl, (hetero)aryl, (hetero)aryl(C₁-C₄)alkyl such as benzyl, (hetero)cycloalkyl or (hetero)cycloalkyl(C₁-C₄)alkyl group, particularly (C₁-C₄)alkyl group; or v) the cationic forms of lysine or guanidine;
the R¹ radicals may comprise, in the same PHA, A⁻ and C⁺ groups in which case the counterions M⁺ and Q⁻ may be absent while ensuring the electroneutrality of the PHA; for example the same PHA may comprise within it a number of carboxylate group(s) “n” and a same number of ammonium group(s) “n” so that the electroneutrality of the PHA is achieved, another scenario is a PHA which comprises two carboxylate groups, and an alkaline-earth metal Ca²⁺ so as to achieve the electroneutrality of the PHA.
the “solvates” represent hydrates and also the combination with linear or branched C₁-C₄ alcohols such as ethanol, isopropanol or n-propanol; the term “chromophore” means a radical derived from a colourless or coloured compound that is capable of absorbing in the UV and/or visible radiation range at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
the term “fluorescent chromophore” means a chromophore which is also capable of re-emitting in the visible range at an emission wavelength λₑₘ of between 400 and 800 nm, and higher than the absorption wavelength. Preferably with a Stokes shift, i.e. the difference between the maximum absorption wavelength and the emission wavelength, of at least 10 nm, Preferably, fluorescent chromophores are derived from fluorescent dyes that are capable of absorbing in the visible range λ_{abs}, i.e. at a wavelength of between 400 and 800 nm, and of re-emitting in the visible range at a λₑₘ of between 400 and 800 nm, More preferentially, fluorescent chromophores are capable of absorbing at a λ_{abs} of between 420 and 550 nm and of re-emitting in the visible range λₑₘ of between 470 and 600 nm;
the term “brightening chromophore” means a chromophore derived from an optical brightening compound or “optical brighteners, optical brightening agents (OBAs)” or “fluorescent brightening agents (FBAs)” or “fluorescent whitening agents (FWAs)”, i.e. which absorbs in the UV radiation range, i.e. at a wavelength λ_{abs} of between 250 and 350 nm, and subsequently re-emits this energy by fluorescence in the visible range at an emission wavelength λₑₘ of between 400 and 600 nm, i.e. wavelengths between blue-violet and blue-green with a maximum in the blue range. Optical brightening chromophores are thus colourless to the eye.
the term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm);
the term “UV-B screening agent” means a chromophore derived from a compound which screens out (or absorbs) UV-B ultraviolet rays at a wavelength of between 280 and 320 nm. Furthermore, unless otherwise indicated, the limits delimiting the extent of a range of values are included in that range of values.

### a) The PHA Copolymer(s)

The composition according to the method of the invention comprises as first ingredient a) one or more PHA copolymers comprising at least two different repeating polymer units chosen from the following units (A) and (B), as defined previously. In one variant, a composition can comprise as first ingredient a) one or more PHA copolymers which contain several different repeating polymer units (A) as defined previously.

The term “copolymer” means that said polymer is derived from the polycondensation of different polymeric repeating units with one another; for example, when said polymer is derived from the polycondensation of polymeric repeating units (A) with (B), the polymeric units (A) are different from the polymeric units (B) (more precisely, R¹ is different from R²). In one variant, when a polymer is derived from the polycondensation of polymeric repeating units (A), the units (A) are different from one another (more precisely, the R¹ groups are different from one repeating unit to the next).

In one variant, a composition, preferably a cosmetic composition, can comprise a) one or more polyhydroxyalkanoate (PHA) copolymers which consist of a succession of the following units (A), and also the optical or geometric isomers thereof, the organic or mineral acid or base salts thereof, and the solvates thereof such as hydrates:

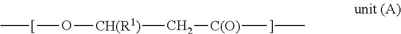

In one variant, a composition, preferably a cosmetic composition as defined previously and hereinbelow comprises a) one or more polyhydroxyalkanoate (PHA) copolymers a) which consist of a succession of units (A) as defined previously in which, when R¹ represents a hydrocarbon-based chain which is saturated and linear comprising 3 to 10 carbon atoms and substituted with one or more carboxyl —C(O)OH groups then said chain is interrupted with heteroatoms or groups a′) to c′) as defined previously.

The composition according to the invention comprises, as first ingredient a), one or more PHA copolymers which comprise at least two different repeating polymer units chosen from units (A) and (B) as defined previously.

The present invention can be a composition, preferably a cosmetic composition, comprising a) one or more polyhydroxyalkanoate (PHA) copolymers a) which comprise at least two different repeating polymer units chosen from the units (A) and (B) below, and also the optical or geometric isomers thereof, the organic or mineral acid or base salts thereof, and the solvates thereof such as hydrates:

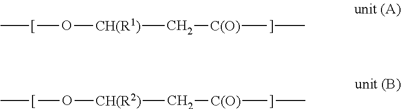

in which polymer units (A) and (B):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms; preferably, the hydrocarbon-based chain is chosen from i) linear or branched (C₅-C₂₆)alkyl, ii) linear or branched (C₆-C₂₈)alkenyl, iii) linear or branched (C₆-C₂₈)alkynyl, iii) preferably the hydrocarbon-based chain is non-cyclic and linear;
said hydrocarbon-based chain being:
   substituted with one or more anionic groups (A⁻), cationic groups (C⁺) and mixtures thereof;
      the A⁻ group(s) being chosen from —C(O)OH, —C(O)O⁻, —SO₃H, —S(O)O⁻, —O—S(O)₂OH, —O—S(O)₂O⁻, —P(O)OH₂, —P(O)O₂⁻, —P(O)₂O⁻, —P(OH)₂, ═P(O)—OH, —P(OH)O⁻, ═P(O)—O⁻, ═P—OH, and ═P—O⁻ and mixtures thereof, the anionic parts comprising one or more cationic counterions M⁺ to achieve the electroneutrality of the PHA, such as i) an alkali metal, ii) an alkaline-earth metal, iii) ammonium RₐR_{b}R_{c}R_{d}N⁺, iv) phosphonium RₐR_{b}R_{c}R_{d}P⁺ with Rₐ representing a hydrogen atom, a (C₁-C₄)alkyl group or (hetero)aryl(C₁-C₄)alkyl group such as benzyl, preferably Rₐ represents a hydrogen atom or a (C₁-C₄)alkyl group; R_{b}, R_{c}, and R_{d}, which may be identical or different, represent a (C₁-C₄)alkyl group optionally substituted with one or more hydroxyl groups, a (hetero)aryl group, a (hetero)aryl(C₁-C₄)alkyl group such as benzyl, a (hetero)cycloalkyl group or a (hetero)cycloalkyl(C₁-C₄)alkyl group, or else R_{b} and R_{c}, or R_{c} and R_{d} represent, with the nitrogen or phosphorus atom which bears them, a saturated or unsaturated, aromatic or non-aromatic heterocycle, particularly a (C₁-C₄)alkyl optionally substituted with one or more hydroxyl groups, v) the cationic forms of lysine or guanidine;
      the C⁺ cationic group(s) being chosen from cationizable amine groups —NR_{b}R_{c}, ammonium groups —N⁺RₐR_{b}R_{c}, phosphonium groups —P⁺RₐR_{b}R_{c}, cationizable amidine groups —C(═NRₐ)—NR_{b}R_{c}, amidinium groups —C(═NRₐ)—N⁺R_{b}R_{c}R_{d}, cationizable guanidine groups —N(Rₐ)—C(═NRₐ)—NR_{b}R_{c}, guanidinium groups —N(Rₐ)—C(═NRₐ)—N⁺R_{b}R_{c}R_{d}, or cationizable imidazolyl groups optionally substituted with one or more Rₐ radicals as defined previously, or imidazolium groups optionally substituted with one or more Rₐ radicals as defined previously, and mixtures thereof to achieve the electroneutrality of the PHA, with Rₐ, R_{b}, R_{c}, and R_{d} as defined previously, the cationic parts comprising one or more anionic counterions Q⁻ to ensure the electroneutrality, such as a halide, a carboxylate such as acetate or glutamate; and optionally;
      it being possible for the R¹ radicals to also be substituted with one or more atoms or groups chosen from: a) halogen such as chlorine or bromine, b) hydroxy, c) thiol, d) (di)(C₁-C₄)(alkyl)amino, e) thiocarboxy, f) (thio)carboxamide —C(O)—N(Rₐ)₂ or —C(S)—N(Rₐ)₂, g) cyano, h) iso(thio)cyanate, i) (hetero)aryl such as phenyl or furyl, and j) (hetero)cycloalkyl, k) cosmetic active agent; l) R—X with R representing a group chosen from α) cycloalkyl such as cyclohexyl, β) heterocycloalkyl such as sugar, preferably monosaccharide such as glucose, γ) (hetero)aryl such as phenyl, m) thiosulfate; X representing a′) O, S, N(R′ₐ) or Si(R′_{b})(R′_{c}), b′) S(O)ᵣ, or (thio)carbonyl, c′) or combinations of a′) with b′) such as (thio)ester, (thio)amide, (thio)urea or sulfonamide; R′ₐ representing a hydrogen atom, or a (C₁-C₄)alkyl group, or aryl(C₁-C₄)alkyl group such as benzyl, preferably Rₐ represents a hydrogen atom; R″_{b} and R′_{c}, which may be identical or different, represent a (C₁-C₄)alkyl or (C₁-C₄)alkoxy group, particularly only one substituent, preferably chosen from a) halogen, and j) such as epoxide and/or
   optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide, preferably ester —O—C(O)— or —C(O)—O— with r being equal to 1 or 2, Rₐ being as defined previously; preferably, Rₐ represents a hydrogen atom, R_{b} and R_{c} being as defined previously;
R² represents a hydrocarbon-based chain as defined previously for R¹; preferably the hydrocarbon-based chain of the R² radical has a carbon number corresponding to the number of carbon atoms of the R¹ radical minus at least one carbon atom is subtracted (notably minus just one carbon atom when for example R¹ is not interrupted and is substituted with a carboxyl or carboxylate group), preferably corresponding to the number of carbon atoms of the R¹ radical minus two carbon atoms; and
   b) optionally water;
   it being understood that:
(A) is different from (B) (i.e R¹, and is different from R²)
the R¹ and R² radicals may comprise, in the same PHA, A⁻ and C⁺ groups in which case the counterions M⁺ and Q⁻ may be absent while ensuring the electroneutrality of the PHA.

According to a particular embodiment, R¹ represents a linear or branched, saturated hydrocarbon-based chain optionally interrupted with S or —O—C(O)— or —C(O)—O⁻, more preferentially S and substituted with one or more ionic groups (A⁻), cationic groups (C⁺) as defined previously and mixtures thereof; preferably a (C₅-C₂₀)alkyl chain substituted with an ionic group (A⁻) or cationic group (C⁺) as defined previously and optionally interrupted with S or —O—C(O)— or —C(O)—O—, more preferentially S, and mixtures thereof. According to a particular embodiment, R² represents a linear or branched, preferably linear, saturated hydrocarbon-based chain that is not substituted and not interrupted, preferably a (C₅-C₂₀)alkyl.

According to a particular embodiment, the molar percentage of units (A) is less than the molar percentage of units (B).

The term “copolymer” means that said polymer is derived from the polycondensation of different polymeric repeating units with one another; for example, when said polymer is derived from the polycondensation of polymeric repeating units (A) with (B), the polymeric units (A) are different from the polymeric units (B) (more precisely, R¹ is different from R²).

According to a particular embodiment of the invention, the PHA copolymer(s) consist of two different repeating polymer units chosen from the units (A) and (B) as defined previously.

More particularly, the PHA copolymer(s) according to the invention comprise the repeating unit of formula (I), and also the optical or geometric isomers thereof, the organic or mineral acid or base salts thereof, and the solvates thereof such as hydrates:

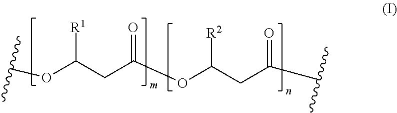

in which formula (I):
R¹ and R² are as defined previously,
m and n are integers greater than or equal to 1; preferably, the sum n+m is inclusively between 450 and 1400; preferably m<n.

According to a particular embodiment, the PHA copolymer(s) of composition a) contain three different repeating polymer units (A), (B) and (C), and preferably consist of three different polymer units (A), (B) and (C) below, and also the optical or geometric isomers thereof and the solvates thereof such as hydrates:

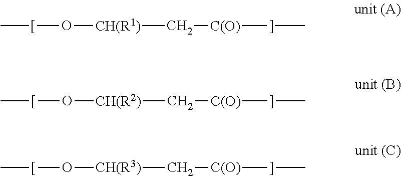

in which polymer units (A), (B) and (C):
R¹ and R² are as defined previously;
R³ represents a cyclic or non-cyclic, linear or branched, saturated or unsaturated hydrocarbon-based group, comprising from 1 to 30 carbon atoms;
optionally substituted with one or more groups chosen from A⁻ and C⁺ as defined previously and/or optionally interrupted with one or more heteroatoms or groups a′) to c′) as defined for R¹; it in particular represents a hydrocarbon-based group chosen from linear or branched (C₁-C₂a)alkyl groups substituted with A⁺ and/or C⁺ as defined previously, preferably, the hydrocarbon-based group has a carbon number corresponding to the number of carbon atoms of the radical R¹ from which at least three carbon atoms are subtracted, preferably corresponding to the number of carbon atoms of the radical R¹ from which at least four carbon atoms are subtracted.
   it being understood that:
(A) is different from (B) and (C), (B) is different from (A) and (C), and (C) is different from (A) and (B); and
preferably, the molar percentage of units (A) is less than the molar percentage of units (B) and less than the molar percentage of units (C) notably if R² represents an alkyl group and/or R³ represent an alkyl group, preferably R³ represents an alkyl group with a carbon number corresponding to the carbon number of R² from which two carbon atoms are subtracted.

According to a particular embodiment, R³ represents a linear or branched, preferably linear, saturated hydrocarbon-based chain that is not substituted and not interrupted, preferably a (C₅-C₂₀)alkyl and more preferentially with a carbon number reduced by 2 units compared to the number of carbon atoms of R².

According to another particular embodiment, R³ represents a linear or branched, preferably linear, saturated hydrocarbon-based chain, said chain being substituted with one or more groups chosen from A⁻ and C⁺ as defined previously and/or optionally interrupted with one or more heteroatoms or groups a′) to c′) as defined for R¹ and more preferentially with a carbon number reduced by 2 units compared to the number of carbon atoms of R¹.

According to a particular embodiment of the invention, the PHA copolymer(s) comprise the repeating unit of formula (II), and also the optical or geometric isomers thereof, the organic or mineral acid or base salts thereof, and the solvates thereof such as hydrates:

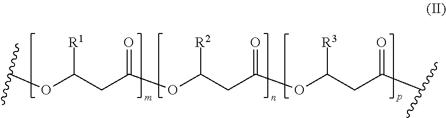

in which formula (II):
R¹, R² and R³ are as defined previously;
m, n and p are integers greater than or equal to 1; preferably, the sum n+m+p is inclusively between 450 and 1400; and
preferably m<n+p, preferably R³ represents an alkyl group with a carbon number corresponding to the carbon number of R² from which 2 carbon atoms have been subtracted.

According to a particular embodiment, the PHA copolymer(s) of composition a) contain four different repeating polymer units (A), (B), (C) and (D), and preferably consist of four different polymer units (A), (B), (C) and (D), below, and also the optical or geometrical isomers thereof, the organic or mineral acid or base salts thereof, and the solvates thereof such as hydrates:

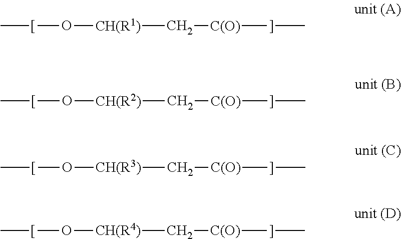

in which polymer units (A), (B), (C) and (D):
R¹, R² and R³ are as defined previously;
R⁴ represents a cyclic or non-cyclic, linear or branched, saturated hydrocarbon-based group comprising from 3 to 30 carbon atoms optionally substituted with one or more groups chosen from A⁻ and C⁺ as defined previously and/or optionally interrupted with one or more heteroatoms or groups a′) to c′) as defined for R′; in particular it represents a hydrocarbon-based group chosen from linear or branched (C₄-C₂₈)alkyl optionally substituted with one or more groups chosen from A⁻ and C⁺ as defined previously and/or optionally interrupted with one or more heteroatoms or groups a′) to c′) as defined for R¹; and it being understood that:
(A) is different from (B), (C) and (D), (B) is different from (A), (C) and (D), (C) is different from (A), (B) and (D), and (D) is different from (A), (B) and (C); and
preferably the molar percentage of units (A) is less than the molar percentage of units (B), preferably R³ represents an alkyl group with a carbon number corresponding to the carbon number of R² from which 2 carbon atoms are subtracted, and R⁴ represents an optionally substituted and/or interrupted alkyl group, an optionally substituted and/or interrupted alkenyl group, or an optionally substituted and/or interrupted alkynyl group with a carbon number corresponding to the carbon number of R¹ from which 2 carbon atoms are subtracted.

According to a particular embodiment, R⁴ represents a linear or branched, preferably linear, saturated hydrocarbon-based chain that is not substituted and not interrupted, preferably a (C₅-C₂₀)alkyl and more preferentially with a carbon number reduced by 4 units compared to the number of carbon atoms of R².

According to a particular embodiment of the invention, the PHA copolymer(s) comprise the repeating unit of formula (III), and also the optical or geometric isomers thereof, the organic or mineral acid or base salts thereof, and the solvates thereof such as hydrates:

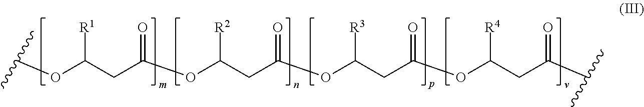

in which formula (III):
R¹, R², R³ and R⁴ are as defined previously;
m, n, p and v are integers greater than or equal to 1; preferably, the sum n+m+p+v is inclusively between 450 and 1400; and
preferably n>m+v; more preferentially n+p>m+v; —preferably R³ represents an alkyl group with a carbon number corresponding to the carbon number of R′ from which 2 carbon atoms are subtracted, and R⁴ represents an alkenyl group optionally substituted with one or more groups chosen from A⁻ and C⁺ as defined previously and/or optionally interrupted with one or more heteroatoms or groups a) to c) as defined for R¹ or alkynyl group substituted with one or more groups chosen from A⁻ and C⁺ as defined previously and/or optionally interrupted with one or more heteroatoms or groups a′) to c′) as defined for R¹ and/or optionally interrupted with a carbon number corresponding to the carbon number of R¹ from which 2 carbon atoms are subtracted.

According to one embodiment, the PHA copolymer(s) of composition a) more particularly contain five different repeating polymer units (A), (B), (C), (D) and (E), and preferably consist of five different polymer units (A), (B), (C), (D) and (E), below, and also the optical or geometric isomers thereof, the organic or mineral acid or base salts thereof, and also the solvates thereof such as hydrates:

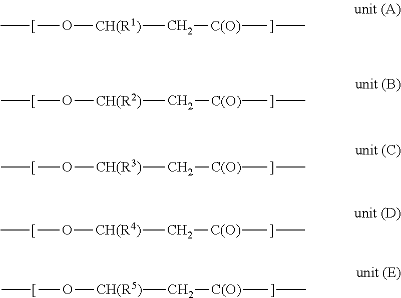

in which polymer units (A), (B), (C), (D) and (E):
R¹, R² R³ and R⁴ are as defined previously; and
R⁵ represents a cyclic or non-cyclic, linear or branched, saturated hydrocarbon-based group comprising from 3 to 30 carbon atoms optionally substituted with one or more groups chosen from A⁻ and C⁺ as defined previously and/or optionally interrupted with one or more heteroatoms or groups a′) to c′) as defined for R¹; it in particular represents a hydrocarbon-based group chosen from linear or branched (C₄-C₂₈)alkyl groups optionally substituted with one or more groups chosen from A⁻ and C⁺ as defined previously and/or optionally interrupted with one or more heteroatoms or groups a′) to c′) as defined for R¹; preferably, the hydrocarbon-based group has a carbon number corresponding to the number of carbon atoms of the radical R⁴ minus at least one carbon atom, preferably corresponding to the number of carbon atoms of the radical R⁴ minus at least two carbon atoms, preferably minus two carbon atoms;
   it being understood that:
(A) is different from (B), (C), (D) and (E); (B) is different from (A), (C), (D) and (E); (C) is different from (A), (B), (D) and (E); (D) is different from (A), (B), (C) and (E); and (E) is different from (A), (B), (C) and (D); and
preferably the molar percentage of units (A) is less than the molar percentage of units (B) and is less than the molar percentage of units (C), notably if R² represents an alkyl group that is not substituted and not interrupted and/or R^{a} represents an alkyl group, and R⁴ and R⁵ represent an alkyl group optionally substituted with one or more groups chosen from A⁻ and C⁺ as defined previously and/or optionally interrupted with one or more heteroatoms or groups a′) to c′) as defined for R¹, alkenyl group optionally substituted with one or more groups chosen from A⁻ and C⁺ as defined previously and/or optionally interrupted with one or more heteroatoms or groups a′) to c′) as defined for R¹ or alkynyl group optionally substituted with one or more groups chosen from A⁻ and C⁺ as defined previously and/or optionally interrupted with one or more heteroatoms or groups a′) to c′) as defined for R¹; preferably R³ represents an alkyl group with a carbon number corresponding to the carbon number of R² from which 2 carbon atoms are subtracted, and R⁴ represents an alkenyl group optionally substituted with one or more groups chosen from A⁻ and C⁺ as defined previously and/or optionally interrupted with one or more heteroatoms or groups a′) to c′) as defined for R¹, or alkynyl group optionally substituted with one or more groups chosen from A⁻ and C⁺ as defined previously and/or optionally interrupted with one or more heteroatoms or groups a′) to c′) as defined for R¹ with a carbon number corresponding to the carbon number of R¹ from which 2 carbon atoms are subtracted, and R⁵ represents an alkenyl group optionally substituted with one or more groups chosen from A⁻ and C⁺ as defined previously and/or optionally interrupted with one or more heteroatoms or groups a′) to c′) as defined for R¹ or alkynyl group optionally substituted with one or more groups chosen from A⁻ and C⁺ as defined previously and/or optionally interrupted with one or more heteroatoms or groups a′) to c′) as defined for R¹ with a carbon number corresponding to the carbon number of R¹ from which 2 carbon atoms are subtracted.

According to a particular embodiment, R⁵ represents a linear or branched, preferably linear, saturated hydrocarbon-based chain that is not substituted and not interrupted, preferably a (C₅-C₂₀)alkyl and more preferentially with a carbon number reduced by 6 units compared to the number of carbon atoms of R².

According to a particular embodiment of the invention, the PHA copolymer(s) comprise the repeating unit of formula (IV), and also the optical or geometric isomers thereof, the organic or mineral acid or base salts thereof, and the solvates thereof such as hydrates:

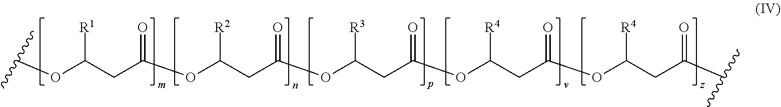

in which formula (IV):
R¹, R², R³, R⁴ and R⁵ are as defined previously;
m, n, p, v and z are integers greater than or equal to 1; preferably, the sum n+m+p+v+z is inclusively between 450 and 1400; and
preferably when R¹ represents an alkyl group substituted with one or more groups chosen from A⁻ and C⁺ as defined previously and/or optionally interrupted with one or more heteroatoms or groups a′) to c′) as defined for R′; alkenyl group substituted with one or more groups chosen from A⁻ and C⁺ as defined previously and/or optionally interrupted with one or more heteroatoms or groups a′) to c′) as defined for R¹; or alkynyl group substituted with one or more groups chosen from A⁻ and C⁺ as defined previously and/or optionally interrupted with one or more heteroatoms or groups a′) to c′) as defined for R¹, R² and R³ represent an alkyl group and the groups R⁴ and R⁵ represent an alkyl group optionally substituted with one or more groups chosen from A⁻ and C⁺ as defined previously and/or optionally interrupted with one or more heteroatoms or groups a′) to c′) as defined for R¹; alkenyl group optionally substituted with one or more groups chosen from A⁻ and C⁺ as defined previously and/or optionally interrupted with one or more heteroatoms or groups a′) to c′) as defined for R¹; or alkynyl group optionally substituted with one or more groups chosen from A⁻ and C⁺ as defined previously and/or optionally interrupted with one or more heteroatoms or groups a′) to c′) as defined for R¹, then n>m+v+z; more preferentially n+p>m+v+z; preferably R³ represents an alkyl group with a carbon number corresponding to the carbon number of R² from which 2 carbon atoms are subtracted, and R⁴ represents an alkenyl group optionally substituted with one or more groups chosen from A⁻ and C⁺ as defined previously and/or optionally interrupted with one or more heteroatoms or groups a′) to c′) as defined for R¹ or alkynyl group optionally substituted with one or more groups chosen from A⁻ and C⁺ as defined previously and/or optionally interrupted with one or more heteroatoms or groups a′) to c′) as defined for R¹ with a carbon number corresponding to the carbon number of R¹ from which 2 carbon atoms are subtracted, and R⁵ represents an alkenyl group optionally substituted with one or more groups chosen from A⁻ and C⁺ as defined previously and/or optionally interrupted with one or more heteroatoms or groups a′) to c′) as defined for R¹ or alkynyl group optionally substituted with one or more groups chosen from A⁻ and C⁺ as defined previously and/or optionally interrupted with one or more heteroatoms or groups a′) to c′) as defined for R¹ with a carbon number corresponding to the carbon number of R¹ from which 4 carbon atoms are subtracted.

According to one embodiment, R¹ radical of the invention is a hydrocarbon-based chain which is 1) either substituted with one or more groups chosen from A⁻ and C⁺ as defined previously, preferably substituted with one or more A⁻ groups, and is not interrupted, 2) or substituted with one or more groups chosen from A⁻ and C⁺ as defined previously, preferably substituted with one or more A⁻ groups, and interrupted with one or more heteroatoms or a′) to c′) groups as defined for R¹, preferably heteroatoms such as O, or S, preferably S, more particularly 2).

According to another embodiment of the invention, the PHA copolymer(s) are such that R¹ represents a hydrocarbon-based chain, notably an alkyl group as defined previously, substituted with one or more (preferably one) groups chosen from A⁻ and C⁺ as defined previously, preferably with an A⁻ group.

According to one particular embodiment of the invention, the pHA copolymer(s) is/are such that R¹ represents a hydrocarbon-based chain, notably i) linear or branched (C₅-C₂₈)alkyl, which is substituted with one or more (preferably one) A⁻ group chosen from —C(O)—OH, —C(O)O⁻, —SO₃H, —S(O)₂O⁻, —O—S(O)₂OH, —O—S(O)₂O⁻, —P(O)OH₂, —P(O)O₂⁻, —P(O)O⁻, —P(OH)₂, ═P(O)—OH, —P(OH)O⁻, ═P(O)—O⁻, ═P—OH, and ═P—O⁻ and mixtures thereof, more preferentially chosen from —C(O)—OH, —C(O)O⁻, —SO₃H, —S(O)₂O⁻, —P(O)OH₂ and —P(O)O₂⁻.

According to a preferred embodiment, A⁻ is chosen from the following groups: carboxylate —C(O)O⁻, sulfonate —S(O)₂O⁻, and phosphonate —P(O)O₂⁻, the anionic parts comprising one or more cationic counterions M⁺ in order to achieve the electroneutrality of the PHA, as defined previously, notably an alkali metal, and alkaline-earth metal, ammonium: RₐR_{b}R_{c}R_{d}N⁺, or phosphonium: RₐR_{b}R_{c}R_{d}P⁺, preferably ammonium, with Rₐ, R_{b}, R_{c} and R_{d} as defined previously, preferably Rₐ represents a hydrogen atom; R_{b} and R_{c}, which may be identical or different, preferably identical, represent a linear or branched, preferably branched (C₁-C₄)alkyl group such as isopropyl or isobutyl, more preferentially isopropyl; and R_{d} represents a linear or branched (C₁-C₄)alkyl group, such as ethyl, more particularly M⁺ represents a diisopropylethylammonium group: (i-Pr)₂N⁺(H)Et.

More preferentially A⁻ is a carboxylate group —C(O)O⁻ M⁺ with M⁺ as defined previously.

According to another particular embodiment of the invention, A represents a group chosen from —C(O)—OH, —C(O)O⁻ M⁺ with M⁺ as defined previously.

According to a particular embodiment of the invention, said substituted hydrocarbon-based chain, notably alkyl, is linear.

According to another particular embodiment of the invention, said substituted hydrocarbon-based chain, notably alkyl, is branched.

According to another particular embodiment of the invention, the hydrocarbon-based chain of the radical R¹ of the invention is substituted and interrupted.

According to a particular embodiment of the invention, the hydrocarbon-based chain (notably an alkyl group as defined previously) of the radical R¹ of the invention is:
substituted with one or more (preferably one) A⁻ groups as defined previously; and
interrupted with one or more (preferably one) atoms or groups chosen from O, S, N(Rₐ) and carbonyl, or combinations thereof such as ester, amide or urea, with Rₐ being as defined previously, preferably Rₐ represents a hydrogen atom; preferably interrupted with one or more atoms chosen from O and S, more preferentially with an O or S, notably S, atom. In particular, the R¹ radical represents an interrupted hydrocarbon-based chain, notably a C₇-C₂₀, more particularly C₈-C₁₈ and even more particularly C₉-C₁₆alkyl.

According to an embodiment of the invention, the composition comprises a) one or more PHA copolymer(s) a) in which R¹ is substituted at the end of the chain on the opposite side from the carbon atom which bears said R¹ radical, with one or more groups chosen from anionic groups (A⁻), and cationic groups (C⁺), preferably a single anionic or cationic group.

According to a particular embodiment of the invention, the PHA copolymer(s) is/are such that R¹ represents a (C₃-C₂₈)alkyl group substituted with one or more groups, preferably just one group, at the chain end (i.e. on the side opposite the point of attachment of the alkyl group to the carbon atom of the —CH— of the unit (A)), said group(s) being chosen from —C(O)O⁻ M⁺ with M⁺ as defined previously and interrupted with a sulfur atom, preferably in the beta position with respect to —C(O)O⁻ M⁺.

According to a particular embodiment of the invention, the PHA copolymer(s) is/are such that R¹ represents a i) linear or branched (C₃-C₂₈)alkyl group which is substituted by one or more (preferably one) A⁻ groups chosen from —C(O)—OH, —C(O)O⁻ M⁺ with M⁺ as defined previously, more preferentially A⁻ is a carboxylate group —C(O)O⁻ M⁺ with M⁺ as defined previously; more particularly R₁ represents a (C₃-C₂₈)alkyl group substituted with one or more, preferably just one group, at the chain end, said group(s) being chosen from —C(O)O⁻ M⁺ with M⁺ as defined previously and interrupted with a sulfur atom, preferably in the beta position with respect to —C(O)O— M+.

According to an embodiment of the invention, said hydrocarbon-based chain R¹ has the following formula —(CH₂)ᵣ—X-(ALK)ᵤ-G with X being as defined previously, in particular representing O, S, N(Rₐ), preferably S, ALK represents a linear or branched, preferably branched (C₁-C₁₀)alkylene, more particularly (C₁-C₈)alkylene, r represents an integer between 6 and 11 inclusive, preferably between 7 and 10, for instance 8; u is 0 or 1; and G represents an A⁻ or C⁺ group as defined previously, preferably A⁻ is for instance —C(O)O⁻ M⁺ with M⁺ as defined previously.

In particular, the PHA copolymer(s) are such that R² is chosen from linear or branched (C₃-C₂₀)alkyl, and linear or branched (C₃-C₂₀)alkenyl, in particular a linear hydrocarbon-based group, more particularly (C₃-C₂)alkyl or (C₃-C₂₀)alkenyl.

According to an embodiment, the hydrocarbon-based group has a number of carbon atoms corresponding to the number of carbon atoms in the radical R¹ minus at least one carbon atom, preferably corresponding to the number of carbon atoms in the radical R¹ minus two carbon atoms.

According to one embodiment of the invention, the PHA copolymer(s) are such that the radical R² is a linear or branched, preferably linear, (C₄-C₆)alkyl group such as n-pentyl or n-hexyl.

According to another embodiment of composition according to the invention, the PHA copolymer(s) of the invention comprise the units (A) bearing an alkyl radical R¹ as defined previously, the units (B) as defined previously and the units (C) bearing a linear or branched (C₆-C₂₀)alkenyl, particularly (C₇-C₁₄)alkenyl and more particularly (C₈-C₁₀)alkenyl radical, which is preferably linear and comprising only one unsaturation at the chain end, in particular, —[CR⁴(R⁵)]_{q}—C(R⁷)—C(R⁷)—R⁸ with R⁴, R⁵, R⁶, R⁷ and R⁸, which may be identical or different, representing a hydrogen atom or a (C₁-C₄)alkyl group such as methyl, preferably a hydrogen atom, and q represents an integer inclusively between 2 and 20, preferably between 3 and 10, more preferentially between 4 and 8 such as 6, such as —[CH₂]_{q}—CH═CH₂ and q represents an integer inclusively between 3 and 8, preferably between 4 and 6, such as 5.

According to one embodiment of the composition according to the invention, the PHA copolymer(s) comprise(s) units (A) bearing an alkyl radical R¹ comprising between 8 and 16 carbon atoms substituted with one or more (preferably one) groups chosen from A⁻ or C⁺ as defined previously, preferably A⁻ as defined previously.

According to one embodiment of the composition according to the invention, the copolymer(s) comprise units (B) bearing a linear or branched, preferably linear, (C₄-C₆)alkyl, preferably (C₄-C₅)alkyl radical R² such as pentyl.

According to another embodiment of composition according to the invention, the PHA copolymer(s) comprise units (A) containing an alkyl radical R¹ as defined previously, units (B) as defined previously and units (C) containing a linear or branched (C₆-C₂₆)alkenyl, particularly (C₂-C₁₄)alkenyl radical and more particularly (C₆-C₁n)alkenyl radical, which is preferably linear, and comprising only one unsaturation at the chain end such as —[CH₂]_{q}—CH═CH₂ and p represents an integer inclusively between 3 and 8, preferably between 4 and 6, such as 5.

According to one particular embodiment of the invention, in the PHA copolymer(s), the unit (A) comprises a hydrocarbon-based chain R¹ which is an alkyl group substituted with A⁻ or C⁺ as defined previously and interrupted in particular with a heteroatom such as S, or alkenyl group substituted with A⁻ or C⁺ as defined previously and interrupted in particular with a heteroatom such as S, or alkynyl group substituted with A⁻ or C⁺ as defined previously and interrupted with a heteroatom such as S, as defined previously, said unit (A) is present in a molar percentage ranging from 0.1% to 80%, more preferentially a molar percentage ranging from 0.5% to 70%, even more preferentially a molar percentage ranging from 1% to 50%, better still a molar percentage ranging from 2% to 40%, a molar percentage ranging from 20% to 40%.

Preferably, when R¹ of the unit (A) is an alkyl hydrocarbon-based chain substituted with A⁻ or C⁺ as defined previously and interrupted in particular with a heteroatom such as S, alkenyl hydrocarbon-based chain substituted with A⁻ or C⁺ as defined previously and interrupted in particular with a heteroatom such as S, or alkynyl hydrocarbon-based chain substituted with A⁻ or C⁺ as defined previously and interrupted in particular with a heteroatom such as S, said unit (A) is present in a molar percentage greater than or equal to 10%, more particularly greater than 20%, preferably between 25% and 35%.

According to another particular embodiment, R¹ is substituted with one or more C⁺ groups notably chosen from cationizable amine groups —NR_{b}R_{c}, ammonium groups —N⁺RₐR_{b}R_{c}, cationizable amidine groups —C(═NRₐ)—NR_{b}R_{c}, amidinium groups —C(═NRₐ)—N⁺R_{b}R_{c}R_{d}, cationizable guanidine groups —N(Rₐ)—C(═NRₐ)—NR_{b}R_{c}, guanidinium groups —N(Rₐ)—C(═NRₐ)—N⁺R_{b}R_{c}R_{d}, or cationizable imidazolyl groups optionally substituted with one or more Rₐ radicals as defined previously, or imidazolium groups optionally substituted with one or more Rₐ radicals as defined previously, and mixtures thereof to achieve the electroneutrality of the PHA, with Rₐ, R_{b}, R_{c} and R_{d} as defined previously, the cationic parts comprising one or more anionic counterions Q⁻ such as a halide.

According to particular embodiment of the invention, in the PHA copolymer(s), the unit (A) is present in a molar percentage ranging from 0.1 mol % to 99 mol %, preferentially in a molar percentage ranging from 0.5 mol % to 80 mol %, more preferentially a molar percentage ranging from 1 mol % to 70 mol %, even more preferentially a molar percentage ranging from 2 mol % to 60 mol %, better still a molar percentage ranging from 5 mol % to 50 mol %; even better still a molar percentage ranging from 10 mol % to 40 mol % and even more preferentially a molar percentage ranging from 25 mol % to 35 mol % of units (A); the unit (B) is present in a molar percentage ranging from 1 mol % to 99.9 mol %; preferentially a molar percentage from 2 mol % to 99.5 mol %, more preferentially a molar percentage from 5 mol % to 90 mol % of units (B); and/or the unit (C) is present in a molar percentage ranging from 0.5 mol % to 30 mol %, preferentially a molar percentage from 1 mol % to 20 mol %, more preferentially from 2 mol % to 10 mol % of units (C).

According to a more particular embodiment of the invention in the PHA copolymer(s), the unit (A) is present in a molar percentage ranging from 0.1% to 80%, more preferentially a molar percentage ranging from 0.5% to 70%, even more preferentially a molar percentage ranging from 1% to 60%, better still a molar percentage ranging from 5% to 50%, a molar percentage ranging from 10% to 40%; the unit (B) is present in a molar percentage ranging from 30% to 99.5%, preferably between 40% and 95%; and the unit (C) is present in a molar percentage ranging from 0 to 30%, preferably between 1% and 25%, more preferentially between 5% and 20% relative to the sum, the unit (D) is present in a molar percentage ranging from 0 to 10%, preferably between 0.1% and 5%, more preferentially between 0.5% and 2% relative to the sum, and the unit (E) is present in a molar percentage ranging from 0 to 10%, preferably between 0.1% and 5%, more preferentially between 0.5% and 2% relative to the sum of the units. Advantageously, the PHA copolymer(s) of the invention comprise from 60 mol % to 80 mol % of units (B).

The values of the molar percentages of the units (A), (B) and (C) of the PHA copolymer(s) are calculated relative to the total number of moles of (A)+(B) if the copolymer(s) do not comprise any additional units (C); otherwise, if the copolymer(s) of the invention contain three different units (A), (B) and (C), then the molar percentage is calculated relative to the total number of moles (A)+(B)+(C); otherwise, if the copolymer(s) of the invention contain four different units (A), (B), (C) and (D), then the molar percentage is calculated relative to the total number of moles (A)+(B)+(C)+(D); otherwise, if the copolymer(s) of the invention contain five different units (A), (B), (C), (D) and (E), then the molar percentage is calculated relative to the total number of moles (A)+(B)+(C)+(D)+(E).

According to a particular embodiment of the invention, the PHA copolymer(s) comprise the following repeating units (A) and (B), and also the optical or geometric isomers thereof, the organic or mineral acid or base salts thereof, and the solvates thereof such as hydrates:

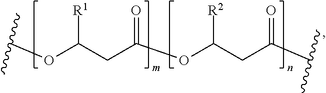

| Compound | R¹ | R² |
|---|---|---|
| (1A) | —(CH₂)ₚ—S—ALK₁—C(O)—OH | —AKL₂ |
| (2A) | —(CH₂)ₚ—S—ALK₁—(A⁻) | —AKL₂ |
| (3A) | —(CH₂)ₚ—S—ALK₁—(C⁺) | —AKL₂ |

p is an integer between 3 and 15;
m and n being as defined previously,
ALK₁ denotes a linear or branched divalent C₁-C₁₅ hydrocarbon-based radical,
ALK₂ denotes a C₃-C₂₀ alkyl radical,
C⁺ and A⁻ are as defined previously.

Preferentially, the PHA copolymer(s) of the invention comprise the following repeating units, and also the optical or geometric isomers thereof, the organic or mineral acid or base salts thereof, and the solvates thereof such as hydrates:

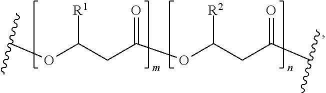

| Compound | R¹ | R² |
|---|---|---|
| (1) | —(CH₂)₈—S—CH(CH₃)—C(O)—OH | n-hexyl |
| (2) | —(CH₂)₈—S—CH(CH₃)—C(O)—O⁻, M⁺ | n-hexyl |

m and n are as defined previously;
M⁺ is a cationic counterion as defined previously;
   In particular, the stereochemistry of the carbon atoms bearing the radicals R¹ and R² is of the same (R) or (S) configuration, preferably of (R) configuration.
   More particularly, the stereochemistry of the carbon atoms bearing the radicals R¹, R² and R³ is of the same (R) or (S) configuration, preferably of (R) configuration.
   More particularly, the stereochemistry of the carbon atoms bearing the radicals R¹, R², R³ and R⁴ is of the same (R) or (S) configuration, preferably of (R) configuration.
   More particularly, the stereochemistry of the carbon atoms bearing the R¹, R², R³, R⁴ and R⁵ radicals is of the same (R) or (S) configuration, preferably of (R) configuration.

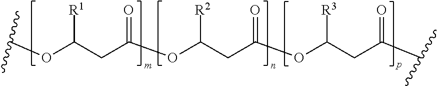

| Compound | R¹ | R² | R³ |
|---|---|---|---|
| (1′) | —(CH₂)₈—S—CH(CH₃)—C(O)—OH | n-hexyl | n-Bu |
| (2′) | —(CH₂)₈—S—CH(CH₃)—C(O)—O⁻, M⁺ | n-hexyl | n-Bu |

with m and n being as defined previously,
M⁺ being as defined previously,
p as defined above;

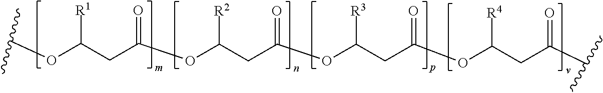

| Compound | R¹ | R² | R³ | R⁴ |
|---|---|---|---|---|
| (1″) | —(CH₂)₈—S—CH(CH₃)—C(O)—OH | n-hexyl | n-Bu | Et |
| (2″) | —(CH₂)₈—S—CH(CH₃)—C(O)—O⁻, M⁺ | n-hexyl | n-Bu | Et |

with m and n being as defined previously,
M⁺ being as defined previously,
p as defined above;
v is an integer greater than or equal to 1.
   The PHA copolymer(s) of the invention preferably have a number-average molecular weight ranging from 50 000 to 500 000, more preferentially from 50 000 to 150 000.

The molecular weight may notably be measured by size exclusion chromatography. A method is described below in the examples.

The PHA copolymer(s) are particularly present in the composition according to the invention in a content ranging from 0.1% to 30% by weight and preferably ranging from 01% to 25% by weight relative to the total weight of the composition.

Advantageously, the PHA copolymer(s) a) are present in a content ranging from 0.1% to 65% by weight, preferably ranging from 0.1% to 60% by weight relative to the total weight of the composition, particularly 1% to 50% by weight, more particularly from 3% to 40% by weight, more preferably from 5% to 35% by weight, even more preferably from 10% to 30% by weight, and better from 15% to 20% by weight relative to the total weight of the composition.

The PHA copolymer(s) preferably have a number-average molecular weight ranging from 50 000 to 500 000, more preferentially from 50 000 to 150 000.

The molecular weight may notably be measured by size exclusion chromatography. A method is described below in the examples.

The copolymer can be present in the composition according to the invention in a content ranging from 01% to 30% by weight, with respect to the total weight of the composition, preferably ranging from 0.1% to 25% by weight.

### Method for Preparing the PHA Copolymer(s):

The methods for preparing the PHA copolymer(s) of the invention are known to those skilled in the art. Mention may notably be made of the use of microbial strains that produce “functionalizable” PHA.

The term “functionalizable” means that the PHA copolymer(s) comprise a hydrocarbon-based chain comprising one or more atoms or groups that are capable of reacting chemically with another reagent—also referred to as “reactive atoms or reactive groups”—to give a Σ covalent bond functionalized by said reagent. The reagent is, for example, a compound comprising at least one nucleophilic group and said functionalized hydrocarbon-based chain comprises at least one electrophilic or nucleofugal atom or group, the nucleophilic group(s) reacting with the electrophilic group(s) to Σ-covalently graft the reagent. The nucleophilic reagent may also react with one or more unsaturations of the alkenyl group(s) to also lead to grafting by covalent bonding of the functionalized hydrocarbon-based chain with said reagent. The addition reaction may also be radical-based, an addition of Markovnikov or anti-Markovnikov type, or nucleophilic or electrophilic substitution. The addition or condensation reactions may or may not take place via a radical route, with or without the use of catalysts or of enzymes, with heating preferably below or equal to 100° C., under a pressure of greater than 1 atm, under an inert atmosphere or under oxygen.

The term “nucleophilic” refers to any atom or group which is electron-donating by an inductive effect +I and/or a mesomeric effect +M. Electron-donating groups that may be mentioned include hydroxyl, thiol and amino groups.

The term “electrophiiic” refers to any atom or group which is electron-withdrawing by an inductive effect −I and/or a mesomeric effect −M. Electron-withdrawing species that may be mentioned include.

The microorganisms producing PHAs of the invention notably bearing a C₃-C₅ hydrocarbon-based chain may be naturally produced by the bacterial kingdom, such as Cyanobacteria of the order of Nostocales (e.g.: Nostoc muscorum, Synechocystis and Synechococcus) but mainly by the Proteobacteria, for example in the class of:
betaproteobacteria, of the order Burkholderiales (Cupriavidus negator synonym Ralstonia eutropha)
alphaproteobacteria, of the order Rhodobacterales (Rhodobacter capsulatus marine and photosynthetic)
gammaproteobacteria, of the order Pseudomonadales of the family Moraxellaceae (Acinetobacter junii).

Among the microorganisms of the bacterial kingdom, the genera Azotobacter, Hydrogenomonas or Chromatium are the most representative of the PHA-producing organisms.

The organisms which naturally produce PHAs notably bearing a C₃-C₅ hydrocarbon-based chain are notably Proteobacteria, such as gammaproteobacteria, and more particularly of the order Pseudomonadales of the family Pseudomonas such as Pseudomonas resinovorans, Pseudomonas putida, Pseudomonas fluorescens, Pseudomonas aeruginosa, Pseudomonas citronellolis, Pseudomonas mendocina, Pseudomonas chlororaphis and preferably Pseudomonas putida GPo1 and Pseudomonas putida KT2440. Preferably Pseudomonas putida and Pseudomonas putida and in particular Pseudomonas putida GPo1 and Pseudomonas putida KT2440.

Certain organisms may also naturally produce PHAs without belonging to the order of Pseudomonadales, such as Comamonas testosteroni which belongs to the class of betaproteobacteria of the order Burkholderiales of the family of Comamonadaceae.

The microorganism producing PHAs according to the invention may also be a recombinant strain if a β-oxidation PHA synthase metabolic pathway is present. The β-oxidation PHA synthase metabolic pathway is mainly represented by four classes of enzymes, EC: 2.3.1 B2, EC: 2.3.1 B3, EC: 2.3.1 B4 and EC: 2.3.1 B5.

The recombinant strain may be of the Bacteria kingdom, e.g.: Escherichia coli or of the Plantae kingdom, e.g.: Chlorella pyrenoidosa: International Journal of Biological Macromolecules, 116, 552-562 “Influence of nitrogen on growth, biomass composition, production, and properties of polyhydroxyalkanoates (PHAs) by microalgae”) or of the Fungi kingdom, e.g. Saccharomyces cerevisiae or Yarrowia lipolytica: Applied Microbiology and Biotechnology 91, 1327-1340 (2011) “Engineering polyhydroxyalkanoate content and monomer composition in the oleaginous yeast Yarrowia lipolytica by modifying the β-oxidation multifunctional protein”).

Use may also be made of genetically modified microorganisms, which may make it possible, for example, to increase the production of PHA, and/or to increase the oxygen consumption capacity, and/or to reduce the autolysis and/or to modify the ratio of the monomers. It is known that, for PHAs, a large portion of the total production cost is devoted to the culture medium and mainly to the substrate/carbon source. Use may thus be made of genetically modified microorganisms using a smaller amount of nutrient (carbon source) for their growth, for example microorganisms that are photoautotrophic by nature, i.e. using light and CO₂ as main energy source.

The copolymer may be obtained in a known manner by biosynthesis, for example with the microorganisms belonging to the genus Pseudomonas, such as Pseudomonas resinovorans, Pseudomonas putida, Pseudomonas fluorescens, Pseudomonas aeruginosa, Pseudomonas citronellolis, Pseudomonas mendocina, Pseudononas chlororaphis and preferably Pseudomonas putida; and with a carbon source which may be a C₂-C₂₀, preferably C₆-C₁₈, carboxylic acid, such as acetic acid, propionic acid, butyric acid, hexanoic acid, heptanoic acid, octanoic acid, nonanoic acid or dodecanoic acid; a saccharide, such as fructose, maltose, lactose, xylose, arabinose, etc.); an n-alkane, such as hexane, octane or dodecane; an n-alcohol, such as methanol, ethanol, octanol or glycerol; methane or carbon dioxide.

The biosynthesis may optionally be performed in the presence of an inhibitor of the β-oxidation pathway, such as acrylic acid, methacrylic acid, propionic acid, cinnamic acid, salicylic acid, pentenoic acid, 2-butynoic acid, 2-octynoic acid or phenylpropionic acid, and preferably acrylic acid.

According to one embodiment, the process for preparing the PHAs of the invention uses microbial cells which produce PHAs via genetically modified microorganisms (GMOs). The genetic modification may increase the production of PHA, increase the oxygen absorption capacity, increase the resistance to the toxicity of solvents, reduce the autolysis, modify the ratio of the PHA comonomers, and/or any combination thereof. In some of these embodiments, the modification of the ratio of the comonomers of the unit (A) increases the amount of predominant monomer versus (B) of the PHA of the invention which is obtained. In another embodiment, the PHA-producing microbial cells reproduce naturally.

As an example of genetically modified PHA-producing microbial strains, mention may be made of Pseudomonas entomophila LAC23 (Adv. Healthcare Mater. 2017, 1601017 (DOI: 10.1002/adhm.201601017.)

As an example of a genetically modified microbial strains producing PHA which is functionalizable or which comprises a reactive group, mention may be made of Pseudomonas entomophila LAC23 (Biomacromolecules, 2014 Jun. 9; 15(6):2310-9. doi: 10.1021/bm500669s)

It is also possible to use wild strains which produce 100% PHO (according to publication dx.doi.org/10.1021/bm2001999|Biomacromolecules, 2011, 12, 2126-2136)

It is also possible to use genetically modified microorganisms which produce phenylvaleric-co-3-hydroxydodecanoic HDD copolymers (Sci. China Life Sci. Shen R., et al., 57 No. 1, (2014) with a strain: Pseudomonas entomophila LAC23.

Nutrients, such as water-soluble salts based on nitrogen, phosphorus, sulfur, magnesium, sodium, potassium and iron, can also be used for the biosynthesis.

The appropriate known conditions of temperature, pH and dissolved oxygen (O_{D}) can be used for the culturing of the microorganisms.

The microorganisms may be cultured according to any known method of culturing, such as in a bioreactor in continuous or batch mode, in fed or unfed mode.

The biosynthesis of the polymers used according to the invention is notably described in the article “Biosynthesis and Properties of Medium-Chain-Length Polyhydroxyalkanoates with Enriched Content of the Dominant Monomer”, Xun Juan et al., Biomacromolecules 2012, 13, 2926-2932, and in patent application WO 2011/069244.

The microbial strains producing PHA which is functionalizable or which comprises a reactive group, as defined previously, are, for example, of the genus Pseudomonas such as P. cichorii YN2, P. citronellolis, P. jessenii, and more generally with species of Pseudomonas putida such as Pseudomonas putida GPo1 (synonym of Pseudomonas oleovorans), P. putida KT2442, P. putida KCTC 2407 and P. putida BM01.

### The Carbon Source(s):

One means for gaining access to the PHAs of the invention is to introduce one or more organic compounds into the culture medium, this or these organic compounds representing a carbon source preferably chosen from alkanes, alkenes, alcohols, carboxylic acids and a mixture thereof.

In one embodiment, the organic compound(s) will preferably be chosen from alcohols, carboxylic acids and a mixture thereof.

The carbon source(s) may be classified as follows:
1) Carbon source via one or more organic compounds introduced into the medium:
2) According to a particular embodiment of the invention, the organic compound(s) are chosen from alcohols, in particular (C₅-C₂₀)alkanols, and/or carboxylic acids, in particular optionally substituted and/or interrupted (C₅-C₂₀)alkanoic acids, notably (C₅-C₂₀)alkanoic acids such as (C₇-C₁₁)alkanoic acids, for instance nonanoic acid or pelargonic acid and/or (C₅-C₂₀)alkenoic acids, notably (C₅-C₂₀)alkenoic acids such as (C₇-C₁₁)alkenoic acids, for instance undecylenic acid, and mixtures thereof.

The carbon source(s) may be classified in three groups:
group A: the organic compound may aid the growth of the productive strain and aid the production of PHA structurally linked to the organic compound.
group B: the organic compound may aid the growth of the strain but does not participate in the production of PHA structurally linked to the organic compound.
group C: the organic compound does not participate in the growth of the strain.

Such microbiological processes are known to those skilled in the art, notably in the scientific literature. Mention may be made of: International Journal of Biological Macromolecules 28, 23-29 (2000); The Journal of Microbiology, 45, No. 2, 87-97, (2007).

According to one variant, the integration of the substrate that is structurally linked to the reactive atom(s) or to the reactive group(s) of the PHA(s) of the invention is introduced directly into the medium as sole carbon source in a medium suitable for microbial growth. (Example: group A for P. putida GPo1: alkenoic acid, notably terminal).

According to another variant, the integration of the substrate that is structurally linked to the reactive atom(s), notably halogen, or to the reactive group(s) of the PHA(s) of the invention is introduced into the medium as carbon source with a second carbon source as co-substrate which is also structurally linked to the PHA, in a medium suitable for microbial growth. (Example: group B for P. putida GPo1: haloalkanoic acids which are preferably terminal, such as terminal bromoalkanoic acids).

According to yet another variant, the integration of the substrate that is structurally linked to the reactive atom(s), notably halogen, or to the reactive group(s) of the PHA(s) of the invention may be introduced directly into the medium as carbon source with a second carbon source as co-substrate which is also structurally linked to the PHA(s) and a third carbon source as co-substrate which is not structurally linked to the PHA(s), in a medium suitable for microbial growth. (Example: group C glucose or sucrose).

In one embodiment, the β-oxidation pathway inhibitor is acrylic acid, 2-butynoic acid, 2-octynoic acid, phenylpropionic acid, propionic acid, trans-cinnamic acid, salicylic acid, methacrylic acid, 4-pentenoic acid or 3-mercaptopropionic acid, preferably acrylic acid.

In one embodiment of the first aspect, the functionalized fatty acid is a functionalized hexanoic acid, functionalized heptanoic acid, functionalized octanoic acid, functionalized nonanoic acid, functionalized decanoic acid, functionalized undecanoic acid, functionalized dodecanoic acid or functionalized tetradecanoic acid.

The functionalization may be introduced by means of an organic compound chosen from precursors of the alcohol and/or carboxylic acid category, notably:
for functionalization of the PHA(s) with a branched alkyl group: see, for example Applied and Environmental Microbiology, 60, No. 9, 3245-325 (1994);
for functionalization of the PHA(s) with a linear alkyl group comprising a terminal cyclohexyl unit: see, for example doi.org/10.1016/SO141-8130(01)00144-1;
for functionalization of the PHA(s) with an unsaturated alkyl group which is preferably terminal: see, for example doi.org/10.1021/bm8005616);
for functionalization of the PHA(s) with a linear alkyl group comprising a halogen preferably at the end of the hydrocarbon-based chain (doi.org/10.1021/ma00033a002);
for functionalization of the PHA(s) with a (hetero)aromatic alkyl group, for example phenyl, benzoyl, phenoxy, see, for example J. Microbiol Biotechnol., 11, 3, 435-442 (2001);
for functionalization of the PHA(s) with a linear alkyl group comprising a heteroatom notably at the end of the hydrocarbon-based chain, see, for example DOI 10.1007/s00253-011-3099-4;
for functionalization of the PHA(s) with a linear alkyl group comprising a cyano function notably at the end of the hydrocarbon-based chain, see, for example doi.org/10.1111/j.1574-6968.1992.tb05839.x;
for functionalization of the PHA(s) with a linear alkyl group comprising an epoxy function notably at the end of the hydrocarbon-based chain, see, for example doi.org/10.1016/S1381-5148(97)00024-2;

The journal International Microbiology 16:1-15 (2013) doi:10.2436/20.1501.01.175 also mentions the majority of the functionalized native PHAs.

In a particular embodiment of the invention, the fatty acid from group A is chosen from 11-undecenoic acid, 10-epoxyundecanoic acid, 5-phenylvaleric acid, citronellol and 5-cyanopentanoic acid, preferably 11-undecenoic acid.

In a particular embodiment of the invention, the fatty acid from group B is chosen from halooctanoic acids such as 8-bromooctanoic acid and 1-bromoundecanoic acid.

In a particular embodiment of the invention, the carbon source from group C is a monosaccharide, preferably glucose.

3) Carbon Source in the Presence of Oxidation Inhibitor Introduced into the Medium:

Another aspect of the invention is the use of the PHA-producing microbial strains in a medium that is suitable for microbial growth, said medium comprising: a substrate which is structurally linked to the PHA(s); at least one carbon source which is not structurally linked to the PHA(s); and at least one oxidation, notably β-oxidation, pathway inhibitor. This allows the growth of the microbial cells to take place in said medium, the microbial cells synthesizing the PHA polymer(s) of the invention; preferably copolymer particularly containing more than 95% of identical units, which has a comonomer ratio of unit (A) and of unit (B) which differs from that obtained in the absence of the β-oxidation pathway inhibitor.

An example of functionalization of PHA copolymers according to the invention starting from a PHA copolymer bearing an unsaturated hydrocarbon-based chain according to scheme 1, in which the compound (b) is prepared from the PHA copolymer bearing an unsaturated hydrocarbon-based chain (a) which undergoes an addition of a nucleophilic reagent YH, it being possible for this reaction to be carried out radically or thermally, preferably radically, notably in the presence of UV radiation, optionally in the presence of radical (photo)initiators such as 2-hydroxy-2-methylpropiophenone, this reaction may be carried out in a (non)polar solvent such as acetone, preferably with stirring at ambient temperature (25° C.) for at least 5 minutes, the molar amount of (b) vs. (a) is between 1 and 10 (molar excess of (b)>(a)), preferably between 2 and 8, more preferentially between 3 and 7, such as 6;

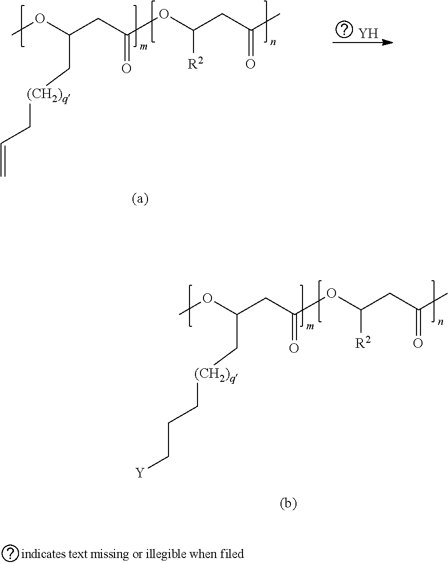

which Scheme 1:
R², m and n are as defined previously;
Y represents a group chosen from —X-(ALK)ₚ-A⁺, M⁺-X-(ALK)ₚ-C⁺, Q⁻ with p being 0 or 1; preferably 1, ALK is as defined previously, preferably represents a (C₁-C₄)alkylene group such as methylene, or ethylene (1,1-ethylene), X being as defined previously, preferably X═S, A⁻ and C⁺ being as defined previously, M⁺ being a cationic counterion as defined previously, and Q⁻ being an anionic counterion as defined previously;
q′ represents an integer inclusively between 2 and 20, preferably between 3 and 10, more preferentially between 4 and 8 such as 6, better still between 3 and 8, preferably between 4 and 6, such as 5.
According to a preferred embodiment of the invention, the process for preparing PHA compounds bearing an R¹ group with a hydrocarbon-based chain substituted with a carboxylate, with Y representing —X-(ALK)ₚ-C(O)O⁻, M⁺ or —X-(ALK)ₚ-C(O)OH, with p being 0 or 1; preferably 1, ALK is as defined previously, preferably represents a (C₁-C₄)alkylene group such as methylene, or ethylene (1,1-ethylene), X being as defined previously, preferably X═S, M⁺ being a cationic counterion as defined previously;
it being understood that if Y represents —X-(ALK)ₚ-C(O)OH, the reaction is followed by a salification reaction with an alkaline agent such as an amine R_{b}R_{b}R_{c}N with R_{b}, R_{b} and R_{c} as defined previously, preferably a tri(C₁-C₄)alkylamine such as N, N-diisopropylethylamine (DIPEA), this reaction may be carried out in solution, notably in a polar solvent such as acetone, preferably at a temperature between 0° C. and 25° C. in particular between 15° C. and 25° C., particularly with stirring, optionally followed by the evaporation of the solvent for example using a rotary evaporator.

Other addition reactions may be performed using double or triple unsaturations such as Michael or Diels-Alder additions, catalytic (notably with Pd or Ni) or non-catalytic hydrogenation reactions, oxidation reactions, which may or may not be controlled, and reactions on electrophiles.

According to one particular embodiment of the invention, the PHA copolymers comprise a linear or branched, saturated hydrocarbon-based chain R¹ which is substituted and/or interrupted with atoms or groups as defined previously for R¹, comprising in total between 5 and 30 carbon atoms, preferably between 6 and 20 carbon atoms, more particularly between seven and 11 carbons and a hydrocarbon-based chain R represents a linear or branched (C₃-C₂₀)alkenyl, particularly (C₁-C₁₄)alkenyl, more particularly (C₇-C₁₀)alkenyl, preferably which is linear and comprises a single unsaturation at the chain end, in particular —[CR⁴(R⁵)]—C(R⁶)═C(R⁷)—R⁸ with R⁴, R⁵, R⁶, R⁷ and R⁸, which may be identical or different, representing a hydrogen atom or a (C₁-C₄)alkyl groups such as methyl, preferably a hydrogen atom and q represents an integer inclusively between 2 and 20, preferably between 3 and 10, more preferentially between 4 and 8, for instance 6, such an —[CH₂]_{q}—CH═CH₂ and q represents an integer inclusively between 3 and 8, preferably between 4 and 6, for instance 5 comprising between 1% and 99%, preferentially between 2% and 50% and even more preferentially between 3% and 40% of unsaturations, and even more particularly between 3% and 30% of unsaturations, better still between 5% and 20% of unsaturations.

A) These unsaturations may be chemically modified via addition reactions, such as radical additions, Michael additions, electrophilic additions, Diels-Alder, halogenation, hydration or hydrogenation reaction, and preferably hydrothiolation reaction with particles, chemical compounds or polymers. In particular, the hydrothiolation reactions may be performed in the presence of a thermal initiator, a redox initiator or a photochemical initiator and of an organic compound bearing a sulfhydryl group, notably chosen from:
linear, branched, cyclic or aromatic alkanethiols including 1 to 14 carbon atoms, such as methane-, ethane-, propane-, pentane- or cyclopentane-thiol, preferably hexane-, cyclohexane-, heptane-, octane-, phenylethane-, 4-tert-butylphenylmethane- or 2-furanmethane-thiol;
organosiloxanes bearing a thiol function, such as (3-mercaptopropyl)trimethoxysilane, (3-mercaptopropyl)methyldimethoxysilane, 2-(triethoxysilyl)ethanethiol or mercaptopropyl-isobutyl-POSS;
thiol-based silicone oils, notably those described in the document DOI: 10.1016/j.actbio.2015.01.020);
thiol-based oligomers or polymers bearing a reactive function, such as an amine, an alcohol, an acid, a halogen, a thiol, an epoxide, a nitrile, an isocyanate, a heteroatom, preferably cysteine, cysteamine, N-acetylcysteamine, 2-mercaptoethanol, 1-mercapto-2-propanol, 8-mercapto-1-octanol, thiolactic acid, thioglycolic acid, 3-mercaptopropionic acid, 11-mercaptoundecanoic acid, polyethylene glycol dithiol, 3-mercaptopropionitrile, 1,3-propanedithiol, 4-cyano-1-butanethiol, 3-chloro-1-propanethiol, 1-thio-β-D-glucose tetraacetate; and
thiols which may be obtained from disulfide reduction, such as phenyl disulfide or furfuryl disulfide.
   Examples of radical initiators that may be mentioned include: tert-butyl peroxy-2-ethylhexanoate, cumene perpivalate, tert-butyl peroxylaurate, benzoyl peroxide, lauroyl peroxide, octanoyl peroxide, di-tert-butyl peroxide, tert-butylcumyl peroxide, dicumyl peroxide, 2,2′-azobisisobutyronitrile, 2,2′-azobis(2-methylbutyronitrile), 2,2′-azobis(2,4-dimethylvaleronitrile), 2,2′-azobis(4-methoxy-2,4-dimethylvaleronitrile), 1,1-bis(tert-butylperoxy)-3,3,5-trimethylcyclohexane, 1,1-bis(tert-butylperoxy)cyclohexane, 1,4-bis(tert-butylperoxycarbonyl)cyclohexane, 2,2-bis(tert-butylperoxy)octane, n-butyl 4,4-bis(tert-butylperoxy)valerate, 2,2-bis(tert-butylperoxy)butane, 1 3-bis(tert-butylperoxyisopropyl)benzene, 2,5-dimethyl-2,5-bis(tert-butylperoxy)hexane, 2,5-dimethyl-2,5-bis(benzoylperoxy)hexane, di-tert-butyl diperoxyisophthalate, 2,2-bis(4,4-di-tert-butylperoxycyclohexyl)propane, di-tert-butyl peroxy-α-methylsuccinate, di-tert-butyl peroxydimethylglutarate, di-tert-butyl peroxyhexahydroterephthalate, di-tert-butyl peroxyazelate, 2,5-dimethyl-2,5-bis(tert-butylperoxy)hexane, diethylene glycol bis(tert-butylperoxycarbonate), di-tert-butyl peroxytrimethyladipate, tris(tert-butylperoxy)triazine, vinyltris(tert-butylperoxy)silane phenothiazine, tetracene, perylene, anthracene, 9,10-diphenylanthracene, thioxanthone, benzophenone, acetophenone, xanthone, fluorenone, anthraquinone, 9,10-dimethylanthracene, 2-ethyl-9,10-dimethyloxyanthracene, 2,6-dimethylnaphthalene, 2,5-diphenyl-1,3,4-oxadiazole, xanthopinacol, 1,2-benzanthracene, 9-nitroanthracene. Each of these initiators may be used alone or in combination with others.
The chemical reactions mentioned previously are known to those skilled in the art. Mention may notably be made of the following documents: Synthesis and preparation of PHAs modified with polyethylene glycol dithiol: 10.1021/acs.biomac.9b00479; Biomacromolecules, 19, 3536-3548 (2018);
Synthesis and preparation of PHAs modified with mercaptohexanol: 10.1021iacs.biomac.8b01257; Biomacromolecules, 20, 2, 645-652 (2019);
Synthesis and preparation of PHAs modified with Hydroxy-, Cinnamic Acid-, Sulfate-, and Zosteric Acid: 10.1021/Bm049962e; Biomacromolecules, 5, 4, 1452-1456 (2004);
Radical addition of methyl methacrylate to a PHOUn: 10.1002/1521-3935(20010701)202:11<2281::AID-MACP2281>3.0.CO; 2-9; Macromolecular Chemistry and Physics, vol. 202, 11, 2281-2286 (2001); Synthesis and preparation of PHAs modified with a polysilsesquioxane (POSS): 10.1016/j.polymer.2005.04.020; Polymer Vol. 46, 14, 5025-5031 (2005);
Grafting of thio-beta-glucose onto unsaturated side chains: 1022-1336/99/0202-0091$17.50+0.50/0; Macromol. Rapid Commun., 20, 91-94 (1999);
   B) These unsaturations may also be chemically modified via controlled or uncontrolled oxidation reactions, for example with permanganates of an alkaline agent (preferably potassium permenganate) which is concentrated or dilute, preferably concentrated, or ozonolysis, and make it possible to obtain PHAs having carboxyl groups in the terminal position of the side chains.
   An example of functionalization of PHA copolymers according to the invention with an aliphatic chain terminated by a carboxyl group (c) or carboxylate group (d) starting from a PHA copolymer bearing an unsaturated hydrocarbon-based chain (a) according to scheme 2,
   which undergoes an uncontrolled oxidation, notably with permanganates of an alkaline agent (preferably potassium permenganate) which is concentrated or dilute, preferably concentrated, and/or in the presence of sodium or potassium carbonate, in water or else in the presence of crown ether, preferably 18-crown-6, in a mixture of halogenated solvent and (C₁-C₄)carboxylica acid such as acetic acid, what else the oxidation is carried out starting from osmium oxide OsO₆, in the presence of a polar protic solvent such as (C₁-C₆)alkanol for instance t-BuOH and oxone, to result in the PHA with an aliphatic chain terminated by a carboxyl group (c), which may be qualified for example with an alkaline agent M⁺OH⁻ to result in the compound (d) of the invention:
   Another example of functionalization of PHA copolymers according to the invention with an aliphatic chain with an ammonium group (f) starting from a PHA copolymer bearing an unsaturated hydrocarbon-based chain (a) according to scheme 2, in which (a) reacts with an amine R_{b}R_{c}NH to result in the PHA copolymer bearing an aliphatic chain with a cationizable amino group (e), it being possible for the latter to then react with RaQ such as a halide of Ra to result in the PHA with an aliphatic chain with an ammonium group (f)

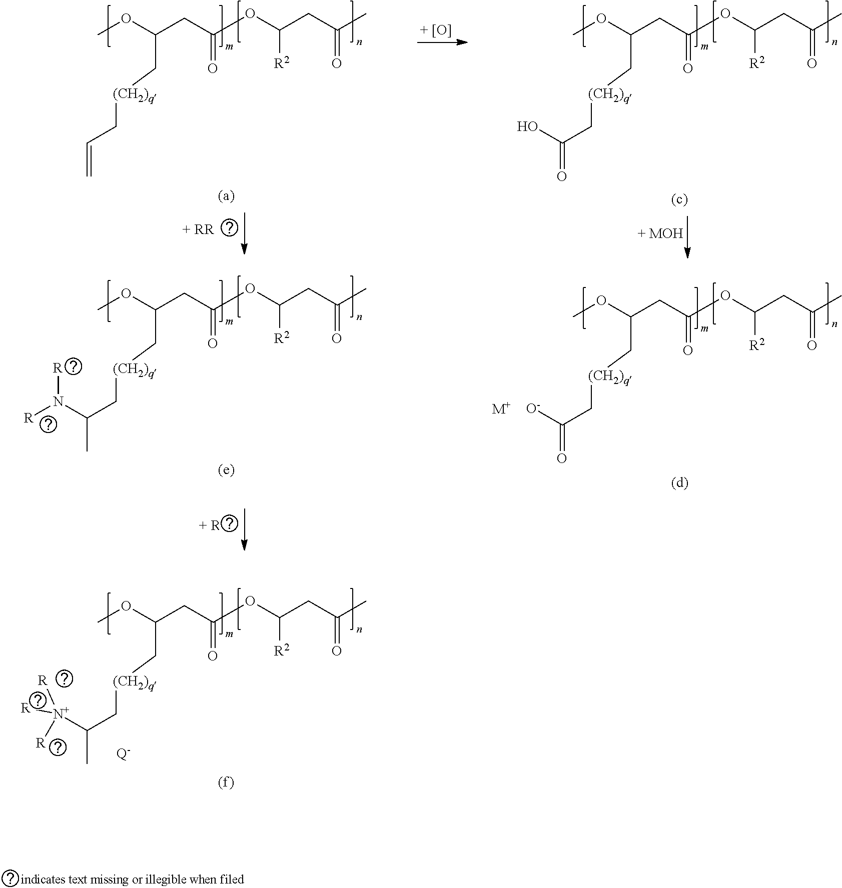

in which Scheme 2:
R², q′, m, and n are as defined previously;
M⁺ being a cationic counterion as defined previously, and
Q⁻ being an anionic counterion as defined previously and Rₐ, R_{b}, and R_{c}, are as defined previously, particularly or a (C₁-C₆)alkyl group optionally substituted with one or more hydroxyl groups, or else R_{b} and R_{c} form, together with the nitrogen atom that bears them, a monocyclic or polycyclic heterocycle, preferably a monocyclic heterocycle, such as pyridino, morpholino, piperazino, piperidino, more preferentially R′ and R″ represent a (hydroxy)(C₁-C₄)alkyl group such as hydroxyethyl.

The chemical reactions mentioned previously are known to those skilled in the art. Mention may notably be made of the following documents:

10.1021/bn049337; Biomacromolecules, vol. 6, 2, 891-896 (2005); 10.1016/S0032-3861(99)00347-X; Polymer, vol. 41, 5, 1703-1709 (2000); 10.1021/ma9714528 et 10.1016/S1381-5148(97)00024-2; Macromolecules, 23, 15, 3705-3707 (1990); 10.1016/30032-3861(01)00692-9; Polymer, vol 43, 4, 1095-1101 (2002); 10.1016/S0032-3861(99)00347-X; Polymer, vol 41, 5, 1703-1709 (2000); and 10.1021/bm025728h; Biomacromolecules, vol 4, 2, 193-195 (2003).

An example of functionalization of PHA copolymers according to the invention starting from a PHA copolymer with an epoxide group (g) according to scheme 3, in which the PHA copolymer with an epoxide group (g) reacts with the nucleophilic reagent YH as defined previously, to result in the PHA according to the invention (h) with a hydrocarbon-based group substituted with a hydroxyl group and a group Y as defined previously; the PHA with an epoxide group (g) may also react with an amine R′R″NH to result in the PHA with an aliphatic chain substituted with a hydroxyl group and with an amino group (i) that can be catalysed in the presence for example of an acid HQ to result in the PHA copolymer with an ammonium group (j) or else the (PHA) copolymer (i) can react with Rₐ-Q such as a halide of Rₐ preferably an alkyl halide to result in the PHA copolymer with an ammonium group (k).

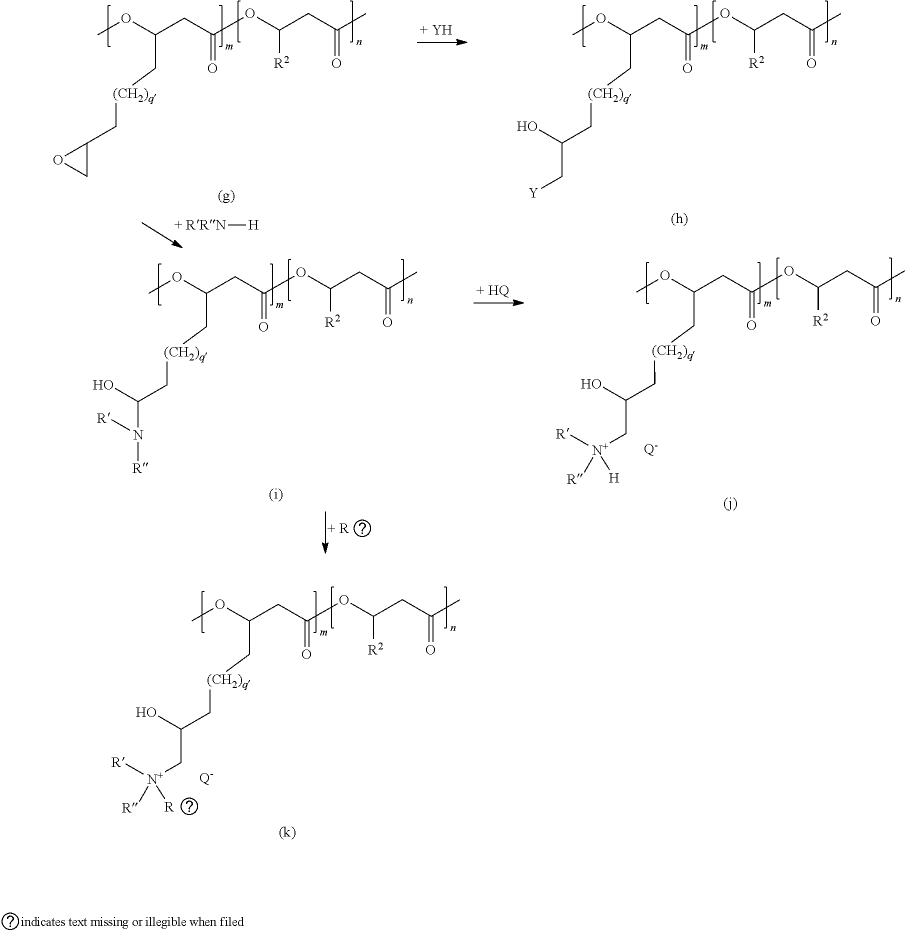

in which Scheme 3: Y, m, n, q′ and R² are as defined in scheme 1, or else Y represents a group R′R″N— with R′R″ representing a hydrogen atom or a (C₁-C₆)alkyl group optionally substituted with one or more hydroxyl groups, or else R′ and R″ form, together with the nitrogen atom that bears them, a monocyclic or polycyclic heterocycle, preferably a mnonocyclic heterocycle, such as pyridino, morpholino, piperazino, piperidino, more preferentially R′ and R″ represent a (hydroxy)(C₁-C₄)alkyl group such as hydroxyethyl.

The epoxide structure may be obtained via a conventional method known to those skilled in the art, whether via biotechnological processes or via chemical processes such as oxidation of unsaturation as mentioned previously. The epoxide group(s) may react with carboxylic acids, maleic anhydrides, amines, alcohols, thiols, isocyanates, all these reagents comprising at least one linear or branched, cyclic or acyclic, saturated or unsaturated C₁-C₂₀ hydrocarbon-based chain; (di)(C₁-C₆)alkylamine, it being possible for the alkyl group(s) to be substituted with one or more hydroxyl groups, such as diethanolamine or dihydroxyethanolamine. It being possible for the amino groups to then be protonated or quaternized in a conventional manner.

Mention may notably be made of the following documents:
Preparation of PHA bearing charges starting with diethanolamine: 10.1021/bm8005616, Biomacromolecules, vol 9, 8, 2091-2096 (2008);
Preparation of PHA bearing charges starting with sodium 3-mercapto-1-propanesulfonate: 10.1021/acs.biomac.9b00870 Biomacromolecules, vol. 20, 9, 3324-3332 (2019);
Preparation of PHA including a native epoxide unit: 10.1016/S1381-5148(97)00024-2); Reactive and Functional Polymers, vol. 34, 1, 65-77 (1997).

An example of functionalization of PHA copolymers according to the invention starting from a PHA copolymer bearing a hydrocarbon-based chain terminated by a nucleofugal group NF (1) according to scheme 4 below, in which the PHA copolymer bearing an aliphatic chain with a nucleofugal group (1) reacts with the nucleophilic reagent YH to result in the PHA (m) bearing an aliphatic chain terminated by a group Y according to the invention:

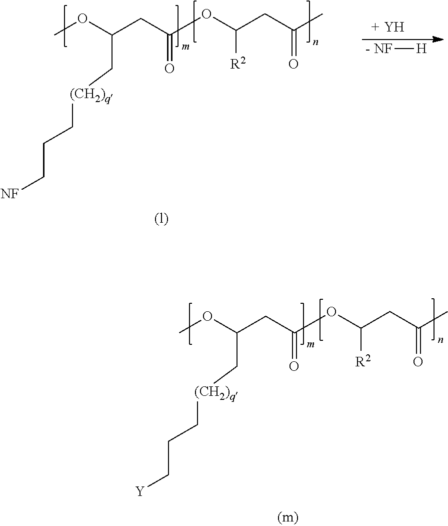

in which Scheme 4 Y, m, n, q′ and R² are as defined in Scheme 1. M corresponds to an organic or inorganic nucleofugal group, which may be substituted with a nucleophilic group; preferably, said nucleophile is a heteroatom which is electron-donating via the +I and/or +M effect such as O, S or N. Preferably, the nucleofugal group M is chosen from halogen atoms such as Br, and mesylate, tosylate or triflate groups. This is a reaction known to those skilled in the art. Mention may be made, for example, of the following document: 10.1016/j.ijbiomac.2016.11.118, International Journal of Biological Macromolecules, vol. 95, 796-808 (2017).

An example of functionalization of PHA copolymers according to the invention starting from a PHA copolymer bearing a hydrocarbon-based chain with a cyano group according to the scheme 5 below, in which the PHA copolymer bearing an aliphatic chain (p) substituted with a cyano or nitrile group, which:
In a first step i), the PHA copolymer bearing a side chain containing a cyano or nitrile group reacts with an organo-alkali metal or organomagnesium compound Y-MgHal, Y-Li or Y-Na, followed by hydrolysis to give the PHA copolymer bearing a side chain containing a group Y grafted with a ketone function (q) according to the invention. The ketone function may be converted into a thioketone by thionation, for example with Ss in the presence of amine, or with Lawesson's reagent. Said thioketone, after complete reduction ii) (for example by Clemmensen reduction), leads to the PHA copolymer bearing a side chain containing a group Y grafted with an alkylene group. Alternatively, said thioketone may undergo a controlled reduction iii) with a conventional reducing agent to give the PHA copolymer bearing a side chain containing a group Y grafted with a hydroxyalkylene group. The cyano group of the starting PHA copolymer can react with water after hydration to give the amide derivative, after iv) after hydrolysis to the carboxyl derivative (t) according to the invention. The latter may then be salified in the presence of an alkaline agent M⁺OH⁻ to result in the copolymer bearing a carboxylate aliphatic chain (u) according to the invention. The cyano group of the starting PHA copolymer may also, after reduction vii), result in the cationizable amine derivative (v), which after amine alkylation reaction and/or salification reaction may result in the copolymer bearing an ammonium aliphatic chain (w) according to the invention. PHA copolymers with a hydrocarbon-based chain bearing a nitrile function are prepared via conventional methods known to those skilled in the art. Mention may be made, for example, of the document: 10.1016/0378-1097(92)90311-B, FEMS Microbiology Letters, vol. 103, 2-4, 207-214 (1992).

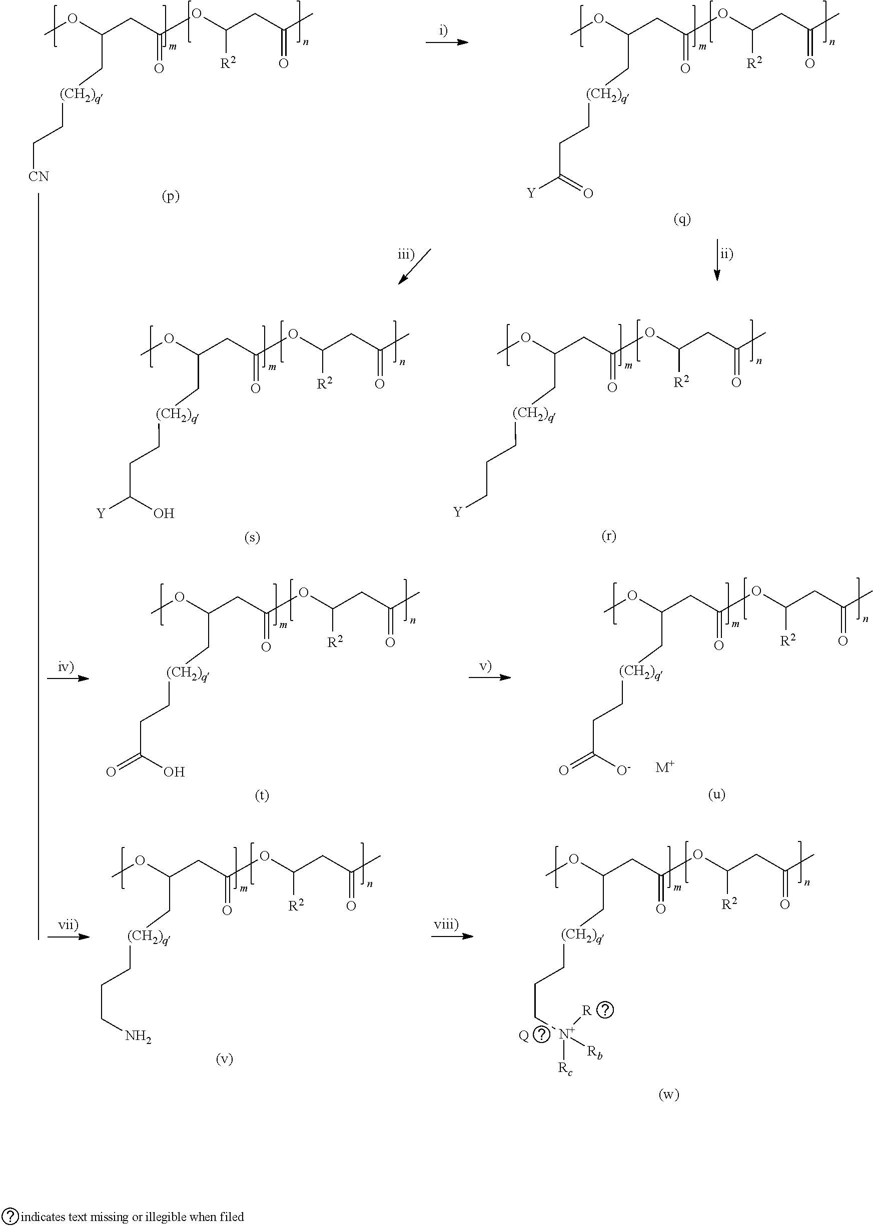

in which Scheme 5: Y, m, n, q′, Y and R², Rₐ, R_{b}, R_{c}, M⁺ and Q⁻ are as defined previously.

Example of functionalization of PHA copolymers according to the invention starting from a PHA copolymer bearing a hydrocarbon-based chain at the chain end, according to Scheme 6 below:

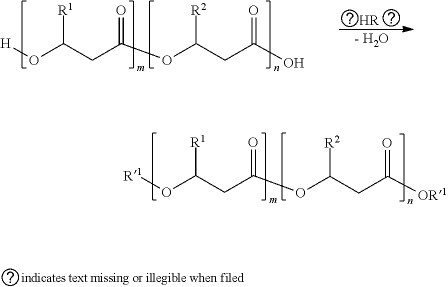

in which Scheme 6: R¹, R², m, n and Y are as defined previously, and R′¹ represents a group chosen from -ALK-A⁻, M⁺ or -ALK-C⁺, Q⁻ with ALK, A⁻, C⁺, M⁺ and Q⁻ as defined previously.

These chain-end grafts onto PHA polymers are known to those skilled in the art. Mention may be made, for example, of the following documents:
Preparation of PHA oligomers by thermal degradation: 10.10211bm0156274; Biomacromolecules, vol. 3, 1, 219-224 (2002);
Preparation of PHA oligomers by transesterification: 10.1021/ma011420r, Macromolecules, vol. 35, 3, 684-689 (2002);
Preparation of PHA oligomers by hydrolysis: 10.101610032-3861(94)90590-8 Polymer, vol. 35, 19, 4156-4162 (1994);
Preparation of PHA oligomers by methanolysis: 10.1021/bm060981t, Biomacromolecules, vol. 8, 4, 1255-1265 (2007).

Mention may also be made of other methods known to those skilled in the art:
Synthesis and characterization of PHA grafted with ascorbic acid: 10.1016/j.ijbiomac.2018.11.052; International Journal of Biological Macromolecules, vol. 123: 7 (2019);
Preparation of PHB-b-PHO copolymers by polycondensation with divinyl adipate catalysed with a lipase: 10.10211bm9011634, Biomacromolecules, vol. 10, 12, 3176-3181 (2009);
Synthesis of PHB-b-PHO copolymers coupled via a diisocyanate junction: 10.1021/ma012223v; Macromolecules, vol. 35, 13, 4946-4950 (2002);
Preparation of PHO oligomers on chitosan by condensation between the carboxylic acid end of the PHO and the amine functions of the chitosan: 10.1002/app.24276; Journal of Applied Polymer Science, vol. 103, 1, (2006);
Transesterification of PHAs with propargyl alcohol in order to produce PHA oligomers that are modifiable by “click” chemistry: 10.1016/j.reactfunctpolym.2011.12.005; Reactive and Functional Polymers, vol. 72, 2, 160-167 (2012);
Preparation of PHO-b-PCL copolymer: 10.1002/mabi.200400104; Macromolecular Bioscience. vol. 4, 11 (2004);
Preparation of PHO-b-PEG copolymer: 10.1002/macp.201000562; Macromolecular Chemistry and Physics; vol. 212, 3, (2010);
Epoxidation of chain-end unsaturation and chain-end grafting of acid: 10.14314/polimery.2017.317; Polimery, vol. 62, 4, 317-322 (2017);
Grafting of organosiloxane unit at chain end onto PHA: 10.1016/j.reactfunctpolym.2014.09.008; Reactive and Functional Polymers, vol. 84, 53-59 (2014).

The combination of grafted PHA copolymers of the invention described previously, according to Scheme 7:

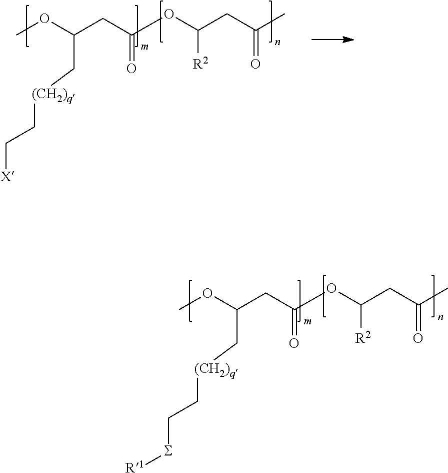

in which Scheme 7 R′¹, R², m, n and Y are as defined previously, and
X′ represents a reactive atom or group that is capable of reacting with an electrophilic E or nucleophilic Nu atom or group to create a Σ covalent bond; if X′ is an electrophilic or nucleofugal group, then it can react with a reagent R′¹-Nu; if X′ is a nucleophilic group Nu, then it can react with R′¹-E to create a Σ covalent bond.

By way of example, the Σ covalent bonds or bonding group that may be generated are listed in the table below, from condensation of electrophiles with nucleophiles:

TABLE 1

| Electropliles E | Nucleophiles Nu | Covalent bonds Σ |
|---|---|---|
| Activated esters* | Amines | Carboxamides |
| Acyl azides** | Amines | Carboxamides |
| Acyl halides | Amines | Carboxamides |
| Acyl halides | Alcohols | Esters |
| Acyl cyanides | Alcohols | Esters |
| Acyl cyanides | Amines | Carboxamides |
| Alkyl halides | Amines | Alkylamines |
| Alkyl halides | Carboxylic acids | Esters |
| Alkyl halides | Thiols | Thioesters |
| Alkyl halides | Alcohols | Ethers |
| Sulfonic acids and salts thereof | Thiols | Thioethers |
| Sulfonic acids and salts thereof | Carboxylic acids | Esters |
| Sulfonic acids and salts thereof | Alcohols | Ethers |
| Anhydrides | Alcohols | Esters |
| Anhydrides | Amines | Carboxamides |
| Aryl halides | Thiols | Thioethers |
| Aryl halides | Amines | Arylamines |
| Aziridines | Thiols | Thioethers |
| Carboxylic acids | Amines | Carboxamides |
| Carboxylic acids | Alcohols | Esters |
| Carbodiimides | Carboxylic acids | N-acylureas |
| Diazoalkanes | Carboxylic acids | Esters |
| Epoxides | Thiols | Thioethers |
| Haloacetamides | Thiols | Thioethers |
| Imide esters | Amines | Amidines |
| Isocyanates | Amines | Ureas |
| Isocyanates | Alcohols | Urethanes |
| Isothiocyanates | Amines | Thioureas |
| Maleimides | Thiols | Thioethers |
| Sulfonic esters | Amines | Alkylamines |
| Sulfonic esters | Thiols | Thioethers |
| Sulfonic esters | Carboxylic acids | Esters |
| Sulfonic esters | Alcohols | Ethers |
| Sulfonyl halides | Amines | Sulfonamides |

*activated esters of general formula —CO-LG with LG representing a leaving group such as oxysuccinimidyl, oxybenzotriazolyl, optionally substituted aryloxy;
**acyl azides can rearrange to give isocyanates

It is also possible, starting with a PHA functionalized on a side chain, to perform chain-end grafting in a second stage as described in Scheme 8. The reverse is also true, in which the chain-end grafting may be performed in a first stage, followed by performing functionalization of a functionalizable pendent side chain in a second stage.

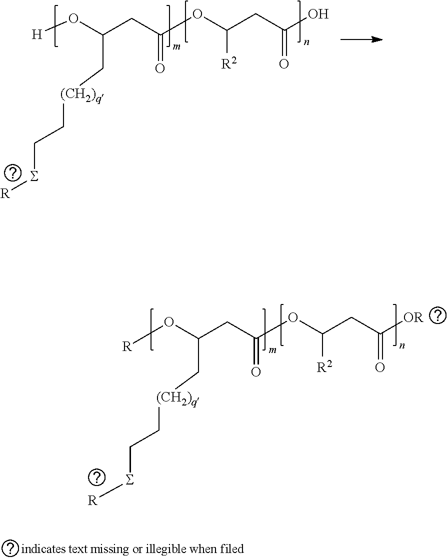

in which Scheme 8: R′¹, R², m, n and Y are as defined previously, and

All these chemical reactions are known to those skilled in the art. Mention may be made, for example, of the following documents:
Synthesis and preparation of PHAs modified with thiol-ene followed by reaction on the new grafted function: 10.1021/ma0304426; Macromolecules, vol. 37, 2, 385-389 (2004);
Grafting of PEG and of PLA onto PHAs functionalized with acids: 10.1002/marc.200900803 and 10.1002/mabi.200390033;
Synthesis and preparation of PHAs modified with polyethylene glycol dithiol: 10.1021/acs.biomac.9b00479.

### b) Water

The composition according to the invention preferably comprises water. The water that is suitable for use in the invention may be tap water, distilled water, spring water, a floral water such as cornflower water and/or a mineral water such as Vittel water, Lucas water or La Roche Posay water and/or a thermal water.

Preferably, water represents the major ingredient by weight of the composition. According to one embodiment, the amount of water by weight is between 20% and 99%, particularly between 30% and 95%, more preferentially between 40% and 90% by weight relative to the total weight of the composition.

According to one embodiment, the composition is an aqueous composition, more preferentially a composition comprising less than 10% by weight of fatty substance relative to the total weight of the composition under consideration, preferably less than 5%, even more preferentially less than 2% and according to one even more preferred embodiment, is free of any fatty substance.

### c) Additional Solvent

The composition may further comprise one or more additional solvents c).

Preferably, the additional solvent(s) are chosen from water-miscible solvents.

According to the present invention, the term “water-miscible solvent” denotes a compound that is liquid at room temperature and water-miscible (miscibility with water of greater than 50% by weight at 25° C. and atmospheric pressure).

The additional solvents that can be used in the composition of the invention may also be volatile. It or they may be polar or nonpolar, protic or aprotic, preferably polar. More particularly, protic and polar.

Among the additional solvents that can be used in the composition according to the invention, mention may notably be made of polar protic solvents such as alcohols, notably lower monoalcohols containing from 2 to 6 carbon atoms, such as ethanol and isopropanol.

According to one embodiment, the composition according to the invention comprises one or more additional solvents, preferably chosen from monoalcohols containing from 2 to 6 carbon atoms, such as ethanol and isopropanol, preferably in an amount of less than 70%, more preferentially less than 20%, even more preferentially less than 10%, more particularly less than 8%, relative to the total weight of water and optionally fatty substance d).

### d) Fatty Substances

According to a particular embodiment of the invention, the composition also comprises one or more fatty substances.

The term “fatty substance” means an organic compound that is insoluble in water at ordinary room temperature (25° C.) and at atmospheric pressure (760 mmHg) (solubility of less than 5%, preferably 1% and even more preferentially 0.1%). They bear in their structure at least one hydrocarbon-based chain including at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

The fatty substance(s) of the invention are of natural or synthetic origin, preferably natural, more preferentially of plant origin. They are different from fatty acids since salified fatty acids constitute soaps which are generally soluble in aqueous media.

According to a particular embodiment of the invention, the composition comprises one or more fatty substances that are not liquid at 25° C. and at atmospheric pressure.

### The Wax(es)

According to a particular embodiment, the composition of the invention comprises one or more waxes.

The term “wax” means a lipophilic compound that is solid at room temperature (25° C.), with a reversible solid/liquid change of state, having a melting point of greater than or equal to 30° C., which may be up to 200° C. and notably up to 120° C.

In particular, the wax(es) that are suitable for use in the invention may have a melting point of greater than or equal to 45° C. and in particular of greater than or equal to 55° C.

The composition according to the invention preferably comprises a content of wax(es) ranging from 0.5% to 30% by weight relative to the total weight of the composition, in particular from 1% to 20% and more particularly from 2% to 15% relative to the total weight of the composition.

According to a particular embodiment of the invention, the composition according to the invention is solid, in particular anhydrous. It may then be in stick form: use will be made of polyethylene microwaxes in the form of crystallites with an aspect ratio at least equal to 2, and with a melting point ranging from 70 to 110° C. and preferably from 70 to 100° C., so as to reduce or even eliminate the presence of strata in the solid composition. These crystallites in needle form and notably the dimensions thereof may be characterized visually according to the following method.

### the Pasty Compound(s)

According to a particular embodiment, the composition according to the invention comprises one or more pasty compounds.

For the purposes of the present invention, the term “pasty compound” means a lipophilic fatty compound that undergoes a reversible solid/liquid change of state, having anisotropic crystal organization in the solid state, and including, at a temperature of 23° C., a liquid fraction and a solid fraction.

According to one embodiment, the composition contains one or more fatty substances d) which are hydrocarbon-based fatty substances that are liquid at 25° C. and atmospheric pressure.

The hydrocarbon-based liquid fatty substance(s) are notably chosen from C₆-C₁₆ hydrocarbons or hydrocarbons comprising more than 16 carbon atoms and up to 60 carbon atoms, preferably between C₆ and C₁₆, and in particular alkanes, oils of animal origin, oils of plant origin, glycerides or fluoro oils of synthetic origin, fatty alcohols, fatty acid and/or fatty alcohol esters, and silicones. In particular, the liquid fatty substance(s) are chosen from non-silicone oils.

It is recalled that, for the purposes of the invention, the fatty alcohols, fatty esters and fatty acids more particularly contain one or more linear or branched, saturated or unsaturated hydrocarbon-based groups comprising 6 to 60 carbon atoms, which are optionally substituted, in particular with one or more hydroxyl groups OH (in particular from 1 to 4 hydroxyl groups). If they are unsaturated, these compounds may comprise one to three unsaturations, preferably from one to three conjugated or unconjugated carbon-carbon double bonds.

As regards the C₆-C₁₆ alkanes, these compounds are linear or branched, and optionally cyclic; preferably, the fatty substance(s) c) of the invention are chosen from linear or branched C₈-C₁₄, more preferentially C₉-C₁₃ and even more preferentially C₉-C₁₂ alkanes. Examples that may be mentioned include hexane, decane, undecane, dodecane, tridecane, and isoparaffins, for instance isohexadecane, isodecane or isododecane. The linear or branched hydrocarbons containing more than 16 carbon atoms may be chosen from liquid paraffins, liquid petroleum jelly, polydecenes, and hydrogenated polyisobutene such as Parleam®.

Among the hydrocarbon-based liquid fatty substances d) having an overall solubility parameter according to the Hansen solubility space of less than or equal to 20 (MPa)^{1/2}, mention may be made of oils, which may be chosen from natural or synthetic, hydrocarbon-based oils, which are optionally fluorinated and optionally branched, alone or as a mixture.

According to a very advantageous embodiment, the composition according to the invention comprises one or more fatty substances which are one or more hydrocarbon-based oils. The hydrocarbon-based oil(s) may be volatile or non-volatile. According to a preferred embodiment of the invention, the fatty substance(s) d) are linear or branched hydrocarbon-based oils, which are volatile, notably chosen from undecane, decane, dodecane, isododecane, tridecane, and a mixture of various volatile oils thereof preferably comprising isododecane in the mixture, or a mixture of undecane and tridecane.

According to another particular embodiment, the liquid fatty substance(s) d) are a mixture of a volatile hydrocarbon-based oil and a non-volatile hydrocarbon-based oil, the mixture of which preferentially comprises dodecane or isododecane as volatile oil.

In particular, the fatty substance(s) d) of the invention are a mixture of C₉-C₁₂ alkanes, preferably of natural origin, the chains of which comprise from 9 to 12 carbon atoms, preferably linear or branched C₉-C₁₂ alkanes. This mixture is notably known under the INCI name C₉-C₁₂ Alkane, CAS 68608-12-8, Vegelight Silk® sold by BioSynthls. This volatile biodegradable mixture of volatile oils is obtained from coconut oil (the viscosity is 0.9-1.1 cSt (40° C.) and it has a flash point of 65° C.).

According to one embodiment, the composition contains only oils that are liquid at 25° C. and atmospheric pressure. According to another embodiment, the composition contains at least 80% of hydrocarbon-based oils that are liquid at 25° C. and atmospheric pressure, which are preferably volatile, more preferentially chosen from isodecane, decane, Cetiol UT® and Vegelight Silk®.

According to another embodiment, the composition may comprise volatile and non-volatile oils, notably in a volatile oil/non-volatile oil ratio of greater than or equal to 4.

According to another embodiment, the composition contains from 0 to 10% of silicone oils, preferably from 0 to 5% of silicone oils.

Volatile silicone oils that may be mentioned include volatile linear or cyclic silicone oils, notably those with a viscosity of less than or equal to 8 centistokes (cSt) (8×10⁻⁶ m²/s), and notably containing from 2 to 10 silicon atoms and in particular from 2 to 7 silicon atoms, these silicones optionally including alkyl or alkoxy groups containing from 1 to 10 carbon atoms. As volatile silicone oils that may be used in the invention, mention may notably be made of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As non-volatile silicone oils, mention may be made of linear or cyclic non-volatile polydimethylsiloxanes (PDMSs); polydimethylsiloxanes including alkyl, alkoxy and/or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and pentaphenyl silicone oils.

The hydrocarbon-based oil may be chosen from:
hydrocarbon-based oils containing from 8 to 14 carbon atoms, and notably:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade names Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in examples 1 and 2 of patent application WO 2008/155059 from the company Cognis, and mixtures thereof, and also mixtures of n-undecane (C11) and of n-tridecane (C13) Cetiol Ultimate® from the company BASF,
short-chain esters (containing from 3 to 8 carbon atoms in total) such as ethyl acetate, methyl acetate, propyl acetate or n-butyl acetate,
hydrocarbon-based oils of plant origin such as triglycerides constituted of fatty acid esters of glycerol, the fatty acids of which may have various chain lengths ranging from C₄ to C₂₄, these chains possibly being linear or branched, and saturated or unsaturated; these oils are notably heptanoic acid or octanoic acid triglycerides, or alternatively wheat germ oil, sunflower oil, grape seed oil, sesame oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, red kuri squash oil, seame oil, pumpkin oil, rapeseed oil, blackcurrant oil, evening primrose oil, millet oil, barley oil, quinoa oil, rye oil, safflower oil, candlenut oil, passionflower oil, musk rose oil or coconut oil; shea butter; or else caprylic/capric acid triglycerides, for instance those sold by the company Stéarinerie Dubois or those sold under the names Miglyol 810®, 812® and 818® by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
esters such as the oils of formula R¹C(O)—O—R² in which R′ represents a linear or branched fatty acid residue including from 1 to 40 carbon atoms and R² represents a, notably branched, hydrocarbon-based chain containing from 1 to 40 carbon atoms, on condition that R¹+R²≥10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters, more preferentially esters of a linear or branched C₈-C₁₀ fatty acid and of a linear or branched C₁₂-C₁₈ fatty alcohol alone or as a mixture with alkanes derived from the complete hydrogenation/reduction of fatty acids obtained from Cocos nucifera (coconut) oil, particularly dodecane or mixtures of cocoyl caprylate/caprate with dodecane; mention may be made of those having the INCI name Coconut alkanes (and) cococaprylate/caprate sold under the name Vegelight 1212LC® by Grant Industries,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

In addition to the hydrocarbon-based liquid fatty substance, the composition according to the invention may comprise a silicone oil. If silicone oil is in the composition according to the invention, it is preferably in an amount which does not exceed 10% by weight relative to the weight of the composition, more particularly in an amount of less than 5% by weight relative to the total weight of the composition.

In particular, the composition comprises at least one hydrocarbon-based liquid fatty substance d) chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, maize oil, arara oil, cottonseed oil, apricot oil, avocado oil, jojoba oil, olive oil, coconut oil or cereal germ oil;
linear, branched or cyclic esters containing more than 6 carbon atoms, notably 6 to 30 carbon atoms; and notably isononyl isononanoate;
   and more particularly esters of formula R^{d}—C(O)—O—R^{e} in which R_{d} represents a higher fatty acid residue including from 7 to 19 carbon atoms and R^{e} represents a hydrocarbon-based chain including from 3 to 20 carbon atoms, such as palmitates, adipates, myristates and benzoates, notably diisopropyl adipate and isopropyl myristate; more preferentially esters of formula R^{d}—C(O)—O—R^{e} in which R_{d} represents a higher fatty acid residue including from 8 to 10 carbon atoms and R^{e} represents a hydrocarbon-based chain including from 12 to 18 carbon atoms:
hydrocarbons and notably volatile or non-volatile linear, branched and/or cyclic alkanes, such as optionally volatile C₅-C₆₀ isoparaffins, such as undecane, dodecane, isododecane, tridecane, Parleam (hydrogenated polyisobutene), isohexadecane, cyclohexane, or Isopars, and mixtures thereof; or alkanes resulting from the complete hydrogenation/reduction of mixtures of fatty acids derived from Cocos nucifera (coconut) oil, such as dodecane, the mixture of C₉-C₁₂ alkanes, the chains of which comprise from 9 to 12 carbon atoms, preferably linear or branched C₉-C₁₂ alkanes, in particular comprising dodecane, or else liquid paraffin, liquid petroleum jelly, or hydrogenated polyisobutylene;
ethers containing 6 to 30 carbon atoms;
ketones containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol;
polyols containing 6 to 30 carbon atoms, such as hexylene glycol; and
mixtures thereof, such as mixtures of esters of linear or branched C₈-C₁₀ fatty acid and C₁₂-C₁₈ fatty alcohol and alkanes resulting from the complete hydrogenation/reduction of mixtures of fatty acids from Cocos nucifera (coconut) oil, in particular dodecane, such as mixtures of cococaprylate/caprate and dodecane; mention may be made of those having the INCI name Coconut alkanes (and) coco-caprylate/caprate sold under the name Vegelight 1212LC® by Grant Industries; or mixtures of C₉-C₁₂ alkanes, the chains of which comprise from 9 to 12 carbon atoms, preferably linear or branched C₉-C₁₂ alkanes, in particular comprising dodecane; mention may be made of the oil mixture having the INCI name C9-12 Alkane, Vegelight Silk® sold by BioSynthls.

Preferably, the composition according to the invention comprises at least one hydrocarbon-based liquid fatty substance d) chosen from:
plant oils formed by fatty acid esters of polyols, in particular triglycerides,
esters of formula R^{d}—C(O)—O—R^{e} in which R^{d} represents a higher fatty acid residue including from 7 to 19 carbon atoms and R^{e} represents a hydrocarbon-based chain including from 3 to 20 carbon atoms, more preferentially esters of formula R^{d}—C(O)—O—R^{e} in which R^{d} represents a higher fatty acid residue including from 8 to 10 carbon atoms and R^{e} represents a hydrocarbon-based chain including from 12 to 18 carbon atoms;
volatile or non-volatile, linear or branched C₈-C₆₀ alkanes, such as isododecane and alkanes resulting from the complete hydrogenation/reduction of mixtures of fatty acids obtained from Cocos nucifera (coconut) oil, in particular dodecane;
volatile or non-volatile, non-aromatic cyclic C₅-C₁₂ alkanes;
ethers containing 7 to 30 carbon atoms;
ketones containing 8 to 30 carbon atoms;
aliphatic fatty monoalcohols containing 12 to 30 carbon atoms, the hydrocarbon-based chain not including any substitution groups; and
mixtures thereof.

Advantageously, the fatty substance(s) d) of the invention, which are notably liquid, are apolar, i.e. formed solely of carbon and hydrogen atoms.

The hydrocarbon-based liquid fatty substance(s) are preferably chosen from hydrocarbon-based oils containing from 8 to 14 carbon atoms, which are in particular volatile, more particularly the apolar oils described previously.

Preferentially, the fatty substance(s) d) of the invention, which are notably liquid, are chosen from alkanes such as dodecane, decane, isododecane, fatty alcohols such as octyldodecanol, esters such as isononyl isononanoate, cocoyl caprylate/caprate and mixtures thereof, more preferentially alkanes.

More particularly, the fatty substance(s) d) of the invention, which are notably liquid, are chosen from linear or branched C₆-C₁₆, preferably C₈-C₁₄, more preferentially C₉-C₁₃ and even more preferentially C₉-C₁₂ alkanes, and even more preferentially the alkanes are volatile, More particularly, the liquid fatty substance(s) d) of the invention are volatile and are chosen from undecane, decane, dodecane, isododecane, tridecane, and a mixture thereof notably comprising dodecane, isododecane or a mixture of undecane and tridecane.

Preferentially, the liquid fatty substance(s) d) of the invention, which are notably liquid, are isododecane.

According to another embodiment of the invention, the fatty substance(s) d) of the invention, which are notably liquid, are a mixture of non-volatile oil(s) and volatile oil(s); preferably, the mixture comprises, as volatile oil, undecane, dodecane, isododecane or tridecane, more preferentially isododecane. A mixture of volatile oil and non-volatile oil that may be mentioned is the mixture of isododecane and of isononyl isononanoate or the mixture of isododecane with isononyl isononanoate.

More preferentially, when the fatty substance(s) are a mixture of volatile oil and of non-volatile oil, the amount of volatile oil is greater than the amount of non-volatile oil.

In particular, in the mixture, the non-volatile oil is a phenyl silicone oil, preferably chosen from pentaphenyl silicone oils.

According to one embodiment, the composition comprises one or more fatty substances, which are notably liquid at 25° C. and at atmospheric pressure, preferably one or more oils, in a content ranging from 0.01% to 99.9% by weight, relative to the total weight of the composition, preferably ranging from 0.1% to 50% by weight, preferably ranging from 0.5% to 20% by weight, preferably ranging from 1% to 5% by weight.

According to one embodiment of the invention, the composition does not comprise any fatty substance d) as defined previously.

According to one embodiment of the invention, the composition according to the invention comprises d) one or more fatty substances that are notably liquid at 25° C. and at atmospheric pressure, b) water and e) one or more surfactants.

### e) Surfactants

According to a particular embodiment of the invention, the composition also comprises e) one or more surfactants, preferably nonionic or ionic surfactants, or mixtures thereof.

The term “surfactant” means a compound which modifies the surface tension between two surfaces. The surfactant(s) e) are amphiphilic molecules, which have two parts of different polarity, one part being lipophilic (which retains fatty substances) which is apolar, the other hydrophilic part (miscible or soluble in water) being polar. The lipophilic part is generally a fatty chain, and the other water-miscible part is polar, and/or protic.

The term “ionic” means anionic, cationic, amphoteric or zwitterionic.

The term “fatty chain” means a linear or branched, saturated or unsaturated hydrocarbon-based chain comprising more than 6 atoms, preferably between 6 and 30 carbon atoms and preferably from 8 to 24 carbon atoms.

According to a first particular embodiment, the composition of the invention contains e) at least one silicone or non-silicone nonionic surfactant.

Among the nonionic surfactants according to the invention, mention may be made, alone or as mixtures, of fatty alcohols, α-diols and alkylphenols, these three types of compound being polyethoxylated, polypropoxylated and/or polyglycerolated and containing a fatty chain including, for example, 8 to 22 carbon atoms, the number of ethylene oxide or propylene oxide groups possibly ranging in particular from 2 to 50 and the number of glycerol groups possibly ranging in particular from 2 to 30. Mention may also be made of ethylene oxide and propylene oxide copolymers, condensates of ethylene oxide and of propylene oxide with fatty alcohols; polyethoxylated fatty amides preferably containing from 2 to 30 mol of ethylene oxide, polyglycerolated fatty amides including on average 1 to 5, and in particular 1.5 to 4, glycerol groups, oxyethylenated fatty acid esters of sorbitan containing from 2 to 30 mol of ethylene oxide, fatty acid esters of sucrose, fatty acid esters of polyethylene glycol, alkyl polyglycosides, N-alkylglucamine derivatives, amine oxides such as (C₁₀-C₁₄)alkylamine oxides or N-acylaminopropylmorpholine oxides.

The surfactant(s) represent(s) in total an amount of less than or equal to 2% by weight of surfactants, relative to the total weight of the composition in question, particularly less than or equal to 1% by weight of surfactants, more particularly less than 0.5% by weight of surfactants, relative to the total weight of the composition in question, preferentially the composition is free of surfactant.

### Form of the Composition:

According to one embodiment of the invention, the composition comprises an aqueous phase. The composition is notably formulated as aqueous lotions or as water-in-oil or oil-in-water emulsions or as multiple emulsions (oil-in-water-in-oil or water-in-oil-in-water triple emulsion (such emulsions are known and described, for example, by C. Fox in “Cosmetics and Toiletries”—November 1986—Vol. 101—pages 101-112)),

The aqueous phase of the composition contains water and in general other water-soluble or water-miscible solvents such as polar and protic solvents as defined previously (see additional solvents).

The composition according to the invention preferably has a pH ranging from 3 to 9, depending on the support chosen.

According to a particular embodiment of the invention, the pH of the composition(s) is neutral or even slightly acidic. Preferably, the pH of the composition is between 6 and 7. The pH of these compositions may be adjusted to the desired value by means of acidifying or basifying agents usually used in cosmetics, or alternatively using standard buffer systems.

The term “basifying agent” or “base” means any agent for increasing the pH of the composition in which it is present. The basifying agent is a Brønsted, Lowry or Lewis base. It may be mineral or organic. Particularly, said agent is chosen from α) aqueous ammonia, b) (bi)carbonate, c) alkanolamines such as monoethanolamine, diethanolamine, triethanolamine and derivatives thereof, d) oxyethylenated and/or oxypropylenated ethylenediamines, e) organic amines, f) mineral or organic hydroxides, g) alkali metal silicates such as sodium metasilicates, h) amino acids, preferably basic amino acids such as arginine, lysine, ornithine, citrulline and histidine, and i) the compounds of formula (F) below:

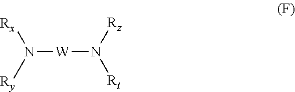

in which formula (F):
W is a divalent C₁-C₆ alkylene radical optionally substituted with one or more hydroxyl groups or a C₁-C₆ alkyl radical, and/or optionally interrupted with one or more heteroatoms such as O or NRᵤ;
Rₓ, R_{y}, R_{z} Rₜ and Rᵤ, which may be identical or different, represent a hydrogen atom or a C₁-C₆ alkyl, C₁-C₆ hydroxyalkyl or C₁-C₆ aminoalkyl radical.

Examples of amines of formula (E) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Among the mineral or organic hydroxides, mention may be made of those chosen from a) hydroxides of an alkali metal, b) hydroxides of an alkaline-earth metal, for instance sodium hydroxide or potassium hydroxide, c) hydroxides of a transition metal, d) hydroxides of lanthanides or actinides, quaternary ammonium hydroxides and guanidinium hydroxide. The mineral or organic hydroxides a) and b) are preferred.

Among the acidifying agents for the compositions used in the invention, examples that may be mentioned include mineral or organic acids, for instance hydrochloric acid, orthophosphoric acid, sulfuric acid, carboxylic acids, for instance acetic acid, tartaric acid, citric acid or lactic acid, or sulfonic acids.

The basifying agents and the acidifying agents as defined previously preferably represent from 0.001% to 20% by weight relative to the weight of the composition containing them, and more particularly from 0.005% to 8% by weight of the composition.

According to one embodiment of the invention, the composition comprises an amount of water of less than or equal to 10% by weight relative to the total weight of the composition, more preferentially comprises an amount of water of less than or equal to 5%, better still less than 2%, even better still less than 0.5% and notably is free of water. Where appropriate, such small amounts of water may notably be introduced by ingredients of the composition that may contain residual amounts thereof.

According to an unpreferred embodiment, the composition does not comprise any water.

Advantageously, the composition according to the invention comprises a physiologically acceptable medium. In particular, the composition is a cosmetic composition.

The term “physiologically acceptable medium” means a medium that is compatible with human keratin materials, for instance the skin, the lips, the nails, the eyelashes, the eyebrows or the hair.

The term “cosmetic composition” means a composition that is compatible with keratin materials, which has a pleasant colour, odour and feel and which does not cause any unacceptable discomfort (stinging, tautness or redness) liable to discourage the consumer from using it.

The term “keratin materials” means the skin (body, face, contour of the eyes, scalp), head hair, the eyelashes, the eyebrows, bodily hair, the nails or the lips.

The composition according to the invention may comprise a cosmetic additive chosen from fragrances, preserving agents, fillers, colouring agents, UV-screening agents, oils, waxes, surfactants, moisturizers, vitamins, ceramides, antioxidants, free-radical scavengers, polymers other than the polyhydroxyalkanoates of the invention, and thickeners.

According to a particular embodiment of the invention, the composition comprises a) at least on colouring agents chosen from pigments, direct dyes and mixtures thereof, preferably a) pigments.

The term “pigment” is understood to mean any pigment, of synthetic or natural origin, which gives colour to keratin materials. The solubility of the pigments in water at 25° C. and at atmospheric pressure (760 mmHg) is less than 0.05% by weight, and preferably less than 0.01%.

They are white or coloured solid particles which are naturally insoluble in the liquid hydrophilic and lipophilic phases usually employed in cosmetics or which are rendered insoluble by formulation in the form of a lake, if appropriate. More particularly, the pigments have little or no solubility in aqueous-alcoholic media.

The pigments that may be used are notably chosen from the organic and/or mineral pigments known in the art, notably those described in Kirk-Othmer's Encyclopedia of Chemical Technology and in Ullmann's Encyclopedia of Industrial Chemistry. Pigments that may notably be mentioned include organic and mineral pigments such as those defined and described in Ullmann's Encyclopedia of Industrial Chemistry “Pigments, Organic”, 2005 Wiley-VCH Verlag GmbH & Co. KGaA, Weinheim 10.1002/14356007.a20 371 and ibid, “Pigments, Inorganic, 1. General” 2009 Wiley-VCH Verlag GmbH & Co. KGaA, Weinheim10.1002/14356007.a20_243.pub3.

These pigments may be in pigment powder or paste form. They may be coated or uncoated.

The pigments may be chosen, for example, from mineral pigments, organic pigments, lakes, special effect pigments such as nacres or glitter flakes, and mixtures thereof.

The pigment may be a mineral pigment. The term “mineral pigment” means any pigment that satisfies the definition in Ullmann's encyclopaedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann's encyclopaedia in the chapter on organic pigments. The organic pigment can in particular be chosen from nitroso, nitro, azo, xanthene, quinoline, anthraquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

In particular, the white or coloured organic pigments may be chosen from carmine, carbon black, aniline black, azo yellow, quinacridone, phthalocyanine blue, sorghum red, the blue pigments codified in the Colour Index under the references CI 42090, 69800, 69825, 73000, 74100, 74160, the yellow pigments codified in the Colour Index under the references CI 11680, 11710, 15985, 19140, 20040, 21100, 21108, 47000, 47005, the green pigments codified in the Colour Index under the references CI 61565, 61570, 74260, the orange pigments codified in the Colour Index under the references CI 11725, 15510, 45370, 71105, the red pigments codified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in patent FR 2 679 771.

According to a particular embodiment of the invention, the pigment(s) used are pigment pastes of organic pigments such as the products sold by the company Hoechst under the name:
Cosmenyl Yellow 10G: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including:
an inorganic core,
at least one binder for fixing the organic pigments to the core, and
at least one organic pigment at least partially covering the core.

The term “lake” is understood to mean dyes adsorbed onto insoluble particles, the assembly thus obtained remaining insoluble during use. The inorganic substrates onto which the dyes are adsorbed are, for example, alumina, silica, calcium sodium borosilicate or calcium aluminium borosilicate, and aluminium. Among the organic dyes, mention may be made of cochineal carmine.

Examples of lakes that may be mentioned include the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green 5 (CI 61 570), D & C Yellow 10 (CI 77 002), D & C Green 3 (CI 42 053), D & C Blue 1 (CI 42 090).

The inorganic substrates onto which the dyes are adsorbed are, for example, alumina, silica, calcium sodium borosilicate or calcium aluminium borosilicate, and aluminium.

Among the dyes, mention may be made of cochineal carmine. Mention may also be made of the dyes known under the following names: D & C Red 21 (CI1 45 380), D & C Orange 5 (CI1 45 370), D & C Red 27 (CI 45 410), D & C Orange 10 (CI1 45 425), 0 & C Red 3 (CI1 45 430), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI1 42 053), D & C Blue 1 (CI1 42 090).

An example of a lake that may be mentioned is the product known under the following name: D & C Red 7 (CI 15 850:1).

The pigment(s) can also be special effect pigments.

The term “special effect pigments” refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of special effect pigments exist: those with a low refractive index, such as fluorescent, photochromic or thermochromic pigments, and those with a higher refractive index, such as nacres or glitter flakes.

Examples of special effect pigments that may be mentioned include nacreous pigments such as titanium mica coated with an iron oxide, mica coated with an iron oxide, mica coated with bismuth oxychloride, titanium mica coated with chromium oxide, titanium mica coated with an organic dye notably of the abovementioned type, and also nacreous pigments based on bismuth oxychloride. They may also be mica particles, at the surface of which are superposed at least two successive layers of metal oxides and/or of organic colorants.

The nacres may more particularly have a yellow, pink, red, bronze, orange, brown, gold and/or coppery colour or tint.

As illustrations of nacres that may be used in the context of the present invention, mention may notably be made of the gold-coloured nacres sold notably by the company Engelhard under the name Gold 222C (Cloisonne), Sparkle gold (Timica), Gold 4504 (Chromalite) and Monarch gold 233X (Cloisonne); the bronze nacres sold notably by the company Merck under the names Bronze fine (17384) (Colorona) and Bronze (17353) (Colorona), by the company Eckart under the name Prestige Bronze and by the company Engelhard under the name Super bronze (Cloisonne); the orange nacres sold notably by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold notably by the company Engelhard under the names Nu-antique copper 340XB (Cloisonne) and Brown CL4509 (Chromalite); the nacres with a copper tint sold notably by the company Engelhard under the name Copper 340A (Timica) and by the company Eckart under the name Prestige Copper; the nacres with a red tint sold notably by the company Merck under the name Sienna fine (17386) (Colorona); the nacres with a yellow tint sold notably by the company Engelhard under the name Yellow (4502) (Chromalite); the red-coloured nacres with a golden tint sold notably by the company Engelhard under the name Sunstone G012 (Gemtone); the black nacres with a golden tint sold notably by the company Engelhard under the name Nu-antique bronze 240 AB (Timica); the blue nacres sold notably by the company Merck under the names Matte blue (17433) (Microna), Dark Blue (117324) (Colorona); the white nacres with a silvery tint sold notably by the company Merck under the name Xirona Silver; and the golden-green pinkish-orange nacres sold notably by the company Merck under the name Indian summer (Xirona), and mixtures thereof.

In addition to nacres on a mica support, multilayer pigments based on synthetic substrates such as alumina, silica, sodium calcium borosilicate or calcium aluminium borosilicate, and aluminium, may be envisaged.

Mention may also be made of interference pigments which are not attached to a substrate, such as liquid crystals (Helicones HC from Wacker) or interference holographic glitter flakes (Geometric Pigments or Spectra f/x from Spectratek). Special effect pigments also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the cosmetic composition according to the present invention is generally between 10 nm and 200 μm, preferably between 20 nm and 80 μm and more preferentially between 30 nm and 50 μm.

The pigments may be dispersed in the product by means of a dispersant.

The term “dispersant” refers to a compound which can protect the dispersed particles from agglomerating or flocculating. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. Said agent may be charged: it may be anionic, cationic, zwitterionic or neutral.

According to a specific embodiment of the invention, the dispersing agents used are chosen from esters of 12-hydroxystearic acid, more particularly, and of C₈ to C₂₀ fatty acid and of polyol, for instance glycerol or diglycerol, such as poly(12-hydroxystearic acid) stearate with a molecular weight of approximately 750 g/mol, such as the product sold under the name of Solsperse 21 000 by Avecia, polyglyceryl-2 dipolyhydroxystearate (CTFA name), sold under the reference Dehymyls PGPH by Henkel, or also polyhydroxystearic acid, such as the product sold under the reference Arlacel P100 by Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the cosmetic composition according to the invention may be surface-treated with an organic agent.

Thus, the pigments surface-treated beforehand that are useful in the context of the invention are pigments which have been completely or partially subjected to a surface treatment of chemical, electronic, electrochemical, mechanochemical or mechanical nature with an organic agent, such as those described notably in Cosmetics and Toiletries, February 1990, Vol. 105, pages 53-64, before being dispersed in the composition in accordance with the invention. These organic agents may be chosen, for example, from amino acids; waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polysaccharides, for example chitosan, cellulose and derivatives thereof; polyethylene; (meth)acrylic polymers, for example polymethyl methacrylates; polymers and copolymers containing acrylate units; proteins; alkanolamines; silicone compounds, for example silicones, polydimethylsiloxanes, alkoxysilanes, alkylsilanes and siloxysilicates; organofluorine compounds, for example perfluoroalkyl ethers; fluorosilicone compounds.

The surface-treated pigments that are useful in the cosmetic composition according to the invention may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used.

The agent for the surface treatment may represent from 0.1% to 50% by weight, preferably from 0.5% to 30% by weight and even more preferentially from 1% to 10% by weight relative to the total weight of the surface-treated pigments.

Preferably, the surface treatments of the pigments are chosen from the following treatments:
a PEG-silicone treatment, for instance the AQ surface treatment sold by LCW;
a chitosan treatment, for instance the CTS surface treatment sold by LCW;
a triethoxycaprylylsilane treatment, for instance the AS surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a lauroyllysine treatment, for instance the LL surface treatment sold by LCW;
a lauroyllysine dimethicone treatment, for instance the LL/SI surface treatment sold by LCW;
a magnesium myristate treatment, for instance the MM surface treatment sold by LCW;
an aluminium dimyristate treatment, such as the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a disodium stearoyl glutamate treatment, for instance the NAI surface treatment sold by Miyoshi;
a dimethicone/disodium stearoyl glutamate treatment, for instance the SA/NAI surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrosiloxane/perfluoroalkyl phosphate treatment, for instance the FS01 surface treatment sold by Daito;
a lauryllysine/aluminium tristearate treatment, for instance the LL-StAl surface treatment sold by Daito;
an octyltriethylsilane treatment, for instance the OTS surface treatment sold by Daito;
an octyltriethylsilane/perfluoroalkyl phosphate treatment, for instance the FOTS surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
a microcrystalline cellulose and carboxymethylcellulose treatment, for instance the AC surface treatment sold by Daito;
a cellulose treatment, for instance the C₂ surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.
The composition in accordance with the present invention may furthermore comprise one or more surface-untreated pigments.
According to one particular embodiment of the invention, the pigment(s) are mineral pigments.
According to another particular embodiment of the invention, the pigment(s) are chosen from nacres.

According to a particular embodiment of the invention, the dispersant is present with organic or inorganic pigments in submicron-sized particulate form.

The term “submicron” or “submicronic” refers to pigments having a particle size that has been micronized by a micronization method and having a mean particle size of less than a micrometre (μm), in particular between 0.1 and 0.9 μm, and preferably between 0.2 and 0.6 μm.

According to one embodiment, the dispersant and the pigment(s) are present in a (dispersant:pigment) amount of between 0.5:1 and 2:1, particularly between 0.75:1 and 1.5:1 or better still between 0.8:1 and 1.2:1.

According to a particular embodiment, the dispersant is suitable for dispersing the pigments and is compatible with a condensation-curable formulation.

The term “compatible” means, for example, that said dispersant is miscible in the oily phase of the composition or of the dispersion containing the pigment(s), and it does not retard or reduce the curing. The dispersing agent is preferably cationic.

The dispersant(s) may therefore have a silicone backbone, such as silicone polyether and dispersants of amino silicone type. Among the suitable dispersants that may be mentioned are:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxylic groups, such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of aminosilicone type and are positively charged.

Mention may also be made of dispersants bearing chemical groups that are capable of reacting with the reagents of the oily phase and are thus capable of improving the 3D network formed from the aminosilicones. For example, dispersants of epoxy silicone pigments can react chemically with the aminosilicone prepolymer amino group(s) to increase the cohesion of the aminosilicone film comprising the pigment(s).

Preferably, the pigment(s) of the invention are chosen from carbon black, iron oxides, notably black iron oxides, and micas coated with iron oxide, triaryimethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides.

The colouring agents may be chosen from direct dyes.

The term “direct dye” means natural and/or synthetic dyes, other than oxidation dyes. These are dyes which will spread superficially over the fibre.

They may be ionic or nonionic, preferably cationic or nonionic, i.e. as sole dyes.

These direct dyes are chosen, for example, from neutral, acidic or cationic nitrobenzene direct dyes, neutral, acidic or cationic azo direct dyes, tetraazapentamethine dyes, neutral, acidic or cationic quinone and in particular anthraquinone dyes, azine direct dyes, triarylmethane direct dyes, azomethine direct dyes and natural direct dyes.

Examples of suitable direct dyes that may be mentioned include azo direct dyes; (poly)methine dyes such as cyanines, hemicyanines and styryl dyes; carbonyl dyes; azine dyes; nitro(hetero)aryl dyes; tri(hetero)arylmethane dyes; porphyrin dyes; phthalocyanine dyes, and natural direct dyes, alone or as mixtures.

Preferentially, the direct dye(s) contain at least one quaternized cationic chromophore or at least one chromophore bearing a quaternized or quaternizable cationic group.

According to a particular embodiment of the invention, the direct dyes comprise at least one quaternized cationic chromophore.

As direct dyes according to the invention, mention may be made of the following dyes: acridines; acridones; anthranthrones; anthrapyrimidines; anthraquinones; azines; (poly)azos, hydrazono or hydrazones, in particular arylhydrazones; azomethines: benzanthrones; benzimidazoles; benzimidazolones; benzindoles; benzoxazoles; benzopyrans; benzothiazoles; benzoquinones; bisazines; bis-isoindolines; carboxanilides; coumarins; cyanines such as azacarbocyanines, diazacarbocyanines, diazahemicyanines, hemicyanines, or tetraazacarbocyanines; diazines; diketopyrrolopyrroles; dioxazines; diphenylamines; diphenylmethanes; dithiazines; flavonoids such as flavanthrones and flavones; fluorindines; formazans; indamines; indanthrones; indigoids and pseudo-indigoids; indophenols; indoanilines; isoindolines; isoindolinones; isoviolanthrones; lactones; (poly)methines such as dimethines of stilbene or styryl type; naphthalimides; naphthanilides; naphtholactams; naphthoquinones; nitro, notably nitro(hetero)aromatics; oxadiazoles; oxazines; perilones; perinones; perylenes; phenazines; phenoxazine; phenothiazines; phthalocyanine; polyenes/carotenoids; porphyrins; pyranthrones; pyrazolanthrones; pyrazolones; pyrimidinoanthrones; pyronines; quinacridones; quinolines; quinophthalones; squaranes; tetrazoliums; thiazines, thioindigo; thiopyronines; triarylmethanes, or xanthenes.

For the cationic azo dyes, mention may be made particularly of those resulting from the cationic dyes described in Kirk-Othmer's Encyclopedia of Chemical Technology, “Dyes, Azo”, J. Wiley & Sons, updated on 19 Apr. 2010.

Among the azo dyes that may be used according to the invention, mention may be made of the cationic azo dyes described in patent applications WO 95/15144, WO 95/01772 and EP-714954.

According to a preferred embodiment of the invention, the direct dye(s) are chosen from cationic dyes known as “basic dyes”.

Among the azo dyes described in the Colour Index International 3rd edition, mention may be made in particular of the following compounds:
Basic Red 22
Basic Red 76
Basic Yellow 57
Basic Brown 16
Basic Brown 17.

Among the cationic quinone dyes, those mentioned in the abovementioned Colour Index International are suitable and, among these, mention may be made, inter alia, of the following dyes:
Basic Blue 22
Basic Blue 99.
Among the azine dyes that are suitable for use, mention may be made of those listed in the Colour Index International, for example the following dyes:
   Basic Blue 17
   Basic Red 2.
Among the cationic triarylmethane dyes that may be used according to the invention, mention may be made, in addition to those listed in the Colour Index, of the following dyes:
   Basic Green 1
   Basic Violet 3
   Basic Violet 14
   Basic Blue 7
   Basic Blue 26.

Mention may also be made of the cationic dyes described in U.S. Pat. No. 5,888,252, EP 1 133 975, WO 03/029 359, EP 860 636, WO 95/01772, WO 95/15144 and EP 714 954. Mention may also be made of those listed in the encyclopaedia “The Chemistry of Synthetic Dyes” by K. Venkataraman, 1952, Academic Press, volumes 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons.

Preferably, the cationic direct dyes are chosen from those resulting from dyes of azo and hydrazono type.

According to a particular embodiment, the direct dyes are cationic azo dyes, described in EP 850 636, FR 2 788 433, ER 920 856, WO 99/48465, FR 2 757 385, ER 850 637, EP 918 053, WO 97/44004, FR 2 570 946, FR 2 285 851, DE 2 538 363, FR 2 189 006, FR 1 560 664, FR 1 540 423, FR 1 567 219, FR 1 516 943, FR 1 221 122, DE 4 220 388, DE 4 137 005, WO 01/66646, U.S. Pat. No. 5,708,151, WO 95/01772, WO 515 144, GB 1 195 386, U.S. Pat. Nos. 3,524,842, 5,879,413, EP 1 062 940, EP 1133 976, GB 738 585, DE 2 527 638, FR 2 275 462, GB 1974-27645, Acta Histochem. (1978), 61(1), 48-52; Tsitologiya (1968), 10(3), 403-5; Zh. Obshch. Khim. (1970), 40(1), 195-202; Ann. Chim. (Rome) (1975), 65(5-6), 305-14; Journal of the Chinese Chemical Society (Taipei) (1998), 45(1), 209-211; Rev. Roum. Chim. (1988), 33(4), 377-83; Text. Res. J. (1984), 54(2), 105-7; Chim. Ind. (Milan) (1974), 56(9), 600-3; Khim. Tekhnol. (1979), 22(5), 548-53; Ger. Monatsh. Chem. (1975), 106(3), 643-8; MRL Bull. Res. Dev. (1992), 6(2), 21-7; Lihua Jianyan, Huaxue Fence (1993), 29(4), 233-4; Dyes Pigm. (1992), 19(1), 69-79; Dyes Pigm. (1989), 11(3), 163-72.

Preferably, the cationic direct dye(s) comprise(s) a quaternary ammonium group; more preferentially, the cationic charge is endocyclic.

These cationic radicals are, for example, a cationic radical:
bearing a (di/tri)(C₁-C₈)alkylammonium exocyclic charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bis-tetrazolium, dihydrothiazolium, imidazopyridinium, imidazolium, indolium, isoquinolium, naphthoirnidazolium, naphthoxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Mention may be made of the hydrazono cationic dyes of formulae (III) and (IV) and the azo cationic dyes of formulae (V) and (VI) below:

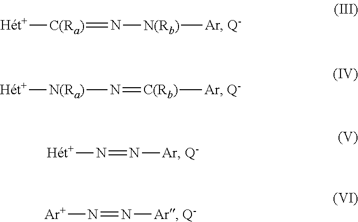

in which formulae (III) to (VI):
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an awl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Rₐ and R_{b}, which may be identical or different, represent a hydrogen atom or a (C₁-C₈)alkyl group, which is optionally substituted, preferentially with a hydroxyl group;
or else the substituent Rₐ with a substituent of Het⁺ and/or R_{b} with a substituent of Ar form, together with the atoms that bear them, a (hetero)cycloalkyl; in particular, Rₐ and R_{b} represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (III) to (VI) as defined previously. More particularly, the cationic direct dyes of formulae (III) to (VI) bearing an endocyclic cationic charge described in patent applications WO 95/15144, WO 95/01772 and EP 714 954. Preferentially the following direct dyes:

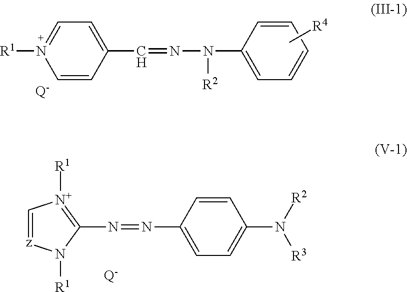

in which formulae (III-1) and (V-1):
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
Z represents a CH group or a nitrogen atom, preferentially CH,
Q⁻ is an anionic counterion as defined previously, in particular a halide, such as chloride, or an alkyl sulfate, such as methyl sulfate or mesityl.

In particular, the dyes of formulae (III-1) and (V-1) are chosen from Basic Red 51, Basic Yellow 87 and Basic Orange 31 or derivatives thereof:

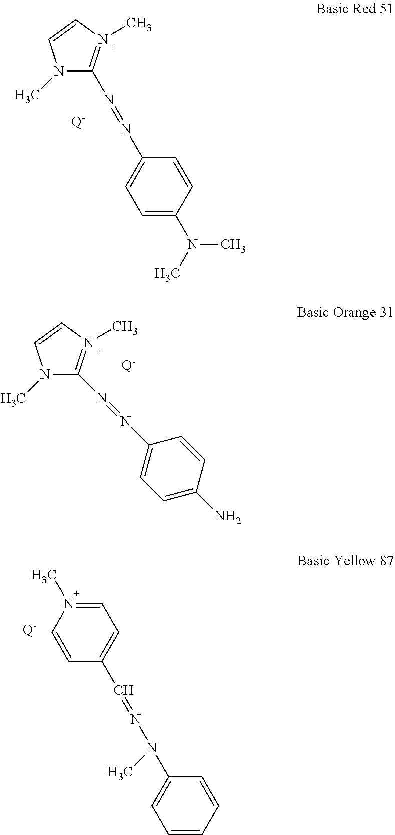

with Q⁻ being an anionic counterion as defined previously, in particular a halide, such as chloride, or an alkyl sulfate, such as methyl sulfate or mesityl.

According to a particular embodiment of the invention, the direct dyes are fluorescent, that is to say that they contain at least one fluorescent chromophore as defined previously.

Fluorescent dyes that may be mentioned include the radicals resulting from the following dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}borons (BODIPY®), diketopyrrolopyrroles, fluorindines, (poly)methines (notably cyanines and styryls/hemicyanines), naphthalimides, naphthanilides, naphthylamines (such as dansyls), oxadiazoles, oxazines, perilones, perinones, perylenes, polyenes/carotenoids, squaranes, stilbenes and xanthenes.

Mention may also be made of the fluorescent dyes described in documents EP 1133975, WO 03/029359, EP 860636, WO 95/01772, WO 95/15144 and EP 714954 and those listed in the encyclopaedia “The Chemistry of Synthetic Dyes” by K. Venkataraman, 1952, Academic Press, volumes 1 to 7, in the “Kirk-Othmer Encyclopedia of Chemical Technology”, in the chapter “Dyes and Dye Intermediates”, 1993, Wiley and Sons, and in various chapters of “Ullmann's Encyclopedia of Industrial Chemistry”, 7th edition, Wiley and Sons, and in the handbook—“A Guide to Fluorescent Probes and Labeling Technologies”, 10th Ed., Molecular Probes/Invitrogen—Oregon 2005, circulated on the Internet or in the preceding printed editions.

According to a preferred variant of the invention, the fluorescent dye(s) are cationic and comprise at least one quaternary ammonium radical, such as those of formula (VII) below:

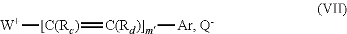

in which formula (VII):
W⁺ represents a cationic heterocyclic or heteroaryl group, particularly comprising a quaternary ammonium optionally substituted with one or more (C₁-C₈)alkyl groups, optionally substituted notably with one or more hydroxyl groups;
Ar representing an aryl group such as phenyl or naphthyl, optionally substituted preferentially with i) one or more halogen atoms such as chlorine or fluorine; ii) one or more (C₁-C₈)alkyl groups, preferably C₁-C₄ alkyl groups, such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino groups or (di)(C₁-C₈)alkylamino groups, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
m′ represents an integer ranging from 1 to 4, in particular m′ is 1 or 2, more preferentially 1;
R_{c} and R_{d}, which may be identical or different, represent a hydrogen atom or an optionally substituted (C₁-C₈)alkyl group, preferentially a C₁-C₄ alkyl group, or alternatively R_{c} contiguous with W′ and/or R_{d} contiguous with Ar form, with the atoms that bear them, a (hetero)cycloalkyl; in particular, R_{c} is contiguous with W⁺ and they form a (hetero)cycloalkyl such as cyclohexyl;
Q⁻ is an organic or mineral anionic counterion as defined previously.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

According to a particular embodiment of the invention, the amount of colouring agents, notably pigments, ranges from 0.5% to 40% and preferably from 1% to 20% relative to the weight of the composition and dispersion comprising them.

Advantageously, the composition according to the invention is a makeup composition, in particular a lip makeup composition, a mascara, an eyeliner, an eye shadow or a foundation.

### The Adjuvants

The composition according to the invention may also comprise one or more fillers, notably in a content ranging from 0.01% to 30% by weight and preferably ranging from 0.01% to 20% by weight relative to the total weight of the composition. The term “fillers” should be understood as meaning colourless or white, mineral or synthetic particles of any shape, which are insoluble in the medium of the composition, irrespective of the temperature at which the composition is manufactured. These fillers notably serve to modify the rheology or texture of the composition.

The composition according to the invention may also contain ingredients commonly used in cosmetics, such as vitamins, thickeners, trace elements, softeners, sequestrants, fragrances, preserving agents, sunscreens, surfactants, antioxidants, agents for combating loss, antidandruff agents and propellants, or mixtures thereof. The composition according to the invention can be in the form of an anhydrous composition, a water-in-oil emulsion or an oil-in-water emulsion, or an aqueous composition.

The term “anhydrous composition” means a composition containing less than 2% by weight of water, or even less than 0.5% of water, and is notably free of water. Where appropriate, such small amounts of water may notably be introduced by ingredients of the composition that may contain residual amounts thereof.

The invention is illustrated in greater detail in the examples that follow. The amounts are indicated as weight percentages.

### EXAMPLES

The PHAs illustrated in the various examples were prepared in 3-litre chemostats and/or 5-litre Fernbach flasks depending on whether or not a β-oxidation pathway inhibitor was used. The isolation of the PHAs is similar for all the examples obtained.

In a first step, the microorganism generates the PHAs which are stored in intracellular granules, the proportion of which varies as a function of the applied conditions such as the temperature or the nature of the culture medium. The generation of PHA granules may or may not be associated with the growth of the microorganism as a function of the nature of the microorganisms. During the second step, the biomass containing the PHAs is isolated, i.e. separated from the fermentation medium, and then dried. The PHAs are extracted from the biomass before being purified, if necessary.

A mixture of saturated and unsaturated carbon sources is, for certain examples, necessary for the stability of the PHA obtained.

TABLE 2

| Carbon source | CAS |
|---|---|
| Caprylic acid (RADIACID 608) | 124-07-2 |
| Nonanoic acid | 112-05-0 |
| Undecylenic acid (10-Undecenoic acid) | 112-38-9 |

TABLE 3

| | Genus and | |
|---|---|---|
| Carbon source | species | Origin |
| Caprylic and undecylenic | Pseudomonas | ATCC ® 47054 ™ |
| acid mixture | putida | |
| Nonanoic and undecylenic | Pseudomonas | ATCC ® 47054 ™ |
| acid mixture | putida | |

### Example 1: PHA Bearing a Side Chain R¹ Representing a Linear 30% Unsaturated n-Octenyl Group and R² Representing an n-Hexyl Group

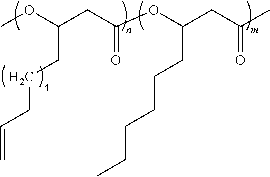

The process for obtaining the PHA of Example 1 is adapted from the article: Fed-batch production of unsaturated medium-chain-length polyhydroxyalkanoates with controlled composition by Pseudomonas putida KT2440, Z. Sun, J. A. Ramsay, M. Guay, B. A. Ramsay, Applied Microbiology Biotechnology, 82. 657-662, 2009.

The microorganism used is Pseudomonas putida KT2440 ATCC® 47054™. The culture method is performed underfed-batch growth axenic conditions with a maintenance solution containing a mixture of carbon sources at a rate μ=0.15 h⁻¹ in a 3 L chemostat containing 2.5 L of culture medium. The flow rate of the maintenance feed pump is proportional to the growth of the microorganism according to Equation 1:

$$St = \frac{{X}_{t}}{{Y}_{X / S}} = \frac{{X}_{0}}{{Y}_{X / S}} {e}^{μ . t}$$

The system is aerated with a flow of 0.5 vvm of air for a dissolved oxygen (DO) setpoint at 30% saturation. The pH is regulated with 15% aqueous ammonia solution. The temperature of the fermentation medium is regulated at 30° C.

### Equipment for the Fed-Batch Growth Fermentation Mode

The fermentation medium is regulated in terms of temperature-pressure of dissolved oxygen and pH (not shown).

The production process is carried out using four different culture media. The first culture medium, defined CM1 “inoculum”, is used for the preparation of the preculture. The second culture medium, defined CM2 “batch”, is used for unfed batch growth of the microorganism with the primary carbon sources in the Fernbach flasks. The third culture medium, defined CM3 “maintenance”, is used for the batch, or maintenance, feeding of the fermentation with the carbon sources of interest at a flow rate calibrated as a function of the growth of the microorganism. A medium CM4 is added at the same time as the medium CM3, with a volume ratio of CM3:MC4=1.25:1).

The composition in grams per litre of the four media is described in Table 6.

TABLE 6

| | CM1 | CM2 | CM3 | |
|---|---|---|---|---|
| | “inoculum” | “batch” | “maintenance” | CM4 |
| (NH4)₂SO4 | 4.7 | 4.7 | / | / |
| Na₂HPO₄•7H₂O | 12 | 9 | / | / |
| KH₂PO₄ | 2.7 | 2.03 | / | / |
| MgSO₄•7H₂O | 0.8 | 1.03 | / | / |
| Nutrient broth | 3 | / | / | / |
| Nonanoic acid | / | 0.7 | 700 | / |
| Undecylenic acid | / | 0.3 | 300 | / |
| Microelement solution | / | 10 | / | / |
| Acrylic acid | / | / | / | 12 |
| Glucose | / | / | / | 240 |

| | | | |
|---|---|---|---|
| 2N NaOH | qs pH 6.8 | / | / |
| MilliQ water | Qsp m = 1000 g | / | / |

The composition of the Nutrient Broth, as mass percentages, is 37.5% beef extract and 62.5% peptone. Reference 233000 DIFCO™.

The composition of the microelement solution in grams per litre is described in Table 7.

TABLE 7

| | | |
|---|---|---|
| FeSO₄•7H₂O | 10.0 | g |
| CaCl₂•2H2O | 3.0 | g |
| ZnSO₄•7H₂O | 2.2 | g |
| MnSO₄•4H₂O | 0.5 | g |
| H₃BO₃ | 0.3 | g |
| CoCl₂•6H₂O | 0.2 | g |
| Na₂MoO₄•2H₂O | 0.15 | g |
| NiCl₂•6H₂O | 0.02 | g |
| CuSO₄•5H₂O | 1.00 | g |
| MilliQ water (or 0.5N HCl) | qs 1000 | g |

100 mL of preculture are prepared by suspending a cryotube containing 1 mL of the strain with 100 mL of “inoculum” culture medium CM1 at a pH adjusted to 6.8 with 2N NaOH in a 250 mL Fernbach flask and then the inoculum is incubated at 30° C. at 150 rpm for 24 h. 1.9 L of CM2 “batch” culture medium placed in a presterilized 3 L chemostat and are inoculated at DO=0.1 with the 100 mL of inoculum. After 4 hours at 30° C. at 850 rpm, introduction of the maintenance culture media (MC3+CM4) is performed, applying the flow rate defined by equation 1.

At the end of the introduction, the biomass is isolated by centrifugation and then washed three times with water. The biomass is dried by lyophilization before being extracted with ethyl acetate for 24 h. The suspension is clarified by filtration on a GF/A filter (Whatman®) and the filtrate composed of PHA in solution in the ethyl acetate, is concentrated by evaporation and then dried under high vacuum at 40° C. to constant mass. The PHA thus obtained was purified twice by dissolving in a minimum of AcOEt and precipitation from a 70/30 v/v ethanol/water mixture.

The PHA of Example 1 was fully characterized by spectroscopic and spectrometric methods and is in accordance with the expected chemical structure.

### Example 2: Functionalization of the PHA Bearing a 30% Unsaturated R=6 and R=8 Linear Side Chain from Example 1 with Thiolactic Acid

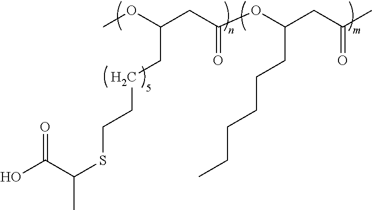

1 g of PHA from Example 1 and 6 equivalents of thiolactic acid per unsaturation (1.2 g), were dissolved in 10 mL of acetone at ambient temperature with stirring. 0.08 equivalents of 2-hydroxy-2-methylpropiophenone (25 mg) were added to the mixture. The reaction medium was then irradiated under a UV lamp (100 W, 365 nm) for at least 10 minutes. The solvent, the excess thiol and the initiator were removed in the oven (24 h, 80° C.) under reduced pressure.

The PHA grafted with the thiolactic acid was characterized by proton NMR. The proton NMR spectrum shows that the characteristic signals of the unsaturations have completely disappeared which confirms a 100% grafting of thiolactic acid to the initially unsaturated chains.

### Example 3: Post-Dispersion in Water of the PHA Bearing a 30% Unsaturated R=6 and R=8 Linear Side Chain Grafted with Thiolactic Acid Obtained in Example 2 with Diisopropylethylamine (DIPEA)

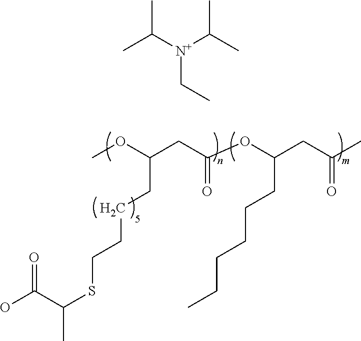

1 g of polymer from Example 2 (PHA functionalized with thiolactic acid) is dissolved in 5 g of acetone to obtain a 20% am solution and then 0.2 g of N,N-diisopropylethylamine (DIPEA) (1.1 eq) are added in order to salify 100% of the acid functions. 5 g of water are then added dropwise undervigourous stirring an UltraTurrax mixer (IKAT10 Basic). Lastly, the acetone is evaporated under reduced pressure using a rotary evaporator.

The polymer obtained at the end the reaction is completely soluble in water (solution containing 20% active material).

### Application Examples

Evaluations in simplex formulations were conducted to show the performance for skin application.

### For One-Action Applications:

A formula containing the solution of polymer, a pigment and a solvent was produced. This formula was applied to a Bioskin type vitro support (elastomeric skin-equivalent support) using a film applicator (wet thickness 100 μm). The deposit was left to dry for 24 h.

The simplex formulation is prepared using a Speed Mixer (2 minutes-3500 rpm).

After drying for 24 h, evaluations of the deposits are performed:

### Resistance to Olive Oil/Water/Sebum

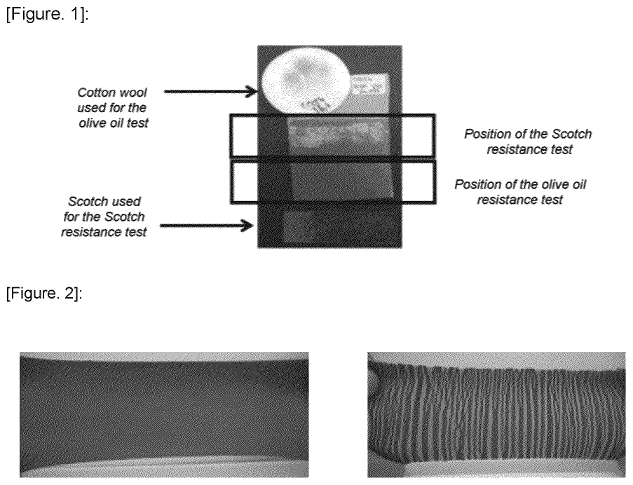

0.5 mL of olive oil (or water or sebum) are applied to the deposit obtained after drying. After 5 minutes, the olive oil (or water or sebum) is removed by wiping 15 times with cotton wool. The deterioration of the film following contact with the olive oil or water or sebum is thus examined—see FIG. 1.

### Fragmentation Test of the Formulations on BioSkin

As for the tests of resistance to olive oil, films are produced on a sample of BioSkin. After drying for one day, the Bioskin sheet is stretched 10 times by hand force. The result may then be observed on the deposit (fragmentation or no fragmentation).

Two response examples are presented in FIG. 2 which is the result of the fragmentation test. On the left: cohesive deposit, no fragmentation/On the right: fragmentation with appearance of deposit breakage under stress

For each simplex composition, the evaluations were carried out after drying. The objective was to observe the improved cosmetic properties with the compounds of the invention. In all cases, the improvement of at least one property was observed for the compounds of the invention compared to a compound outside of the invention.

The evaluation was made in the following manner:
+++: cosmetic property evaluated as very effective
++: cosmetic property evaluated as moderately effective
+: cosmetic property evaluated as not very effective
0: cosmetic property evaluated as ineffective

TABLE 8
Composition A (Invention)

| Ingredients | % AM |
|---|---|
| Aqueous post-dispersion containing 20% | 97 |
| AM of polymer PHA from Example 3 | |
| Red iron oxide | 3 |

TABLE 9
Composition B (comparative)

| Ingredients | % AM |
|---|---|
| Solution of the polymer from Example | 97 |
| 1 at 20% am in isododecane | |
| Red iron oxide | 3 |

The evaluation results are summarized in the table below:

TABLE 9

| | | Resistance | Resistance | Resistance |
|---|---|---|---|---|
| | Fragmentation | to water | to olive oil | to sebum |
| Composition B | +++ | +++ | ++ | + |
| (comparative) | | | | |
| Composition A | +++ | +++ | +++ | +++ |
| (Invention) | | | | |

After application of the compositions of the invention on keratin materials such as synthetic skin, it appears that the deposits obtained are resistant to daily chemical (water/olive oil/sebum) and mechanical (fragmentation) attacking factors.

### Hair Application:

### Hairstyling Evaluation Protocol

A lock of 1 g of 90% NG hair is wound around a brush.

2 g of a solution containing the PHA polymer from Example 3 at 10% in water are sprayed onto the lock. The lock was weighed before and after application. About 0.5 g of 10% solution is deposited on the lock.

The lock is measured after application and 24 hours after application (lock left at room temperature).
A comparison was made with a lock onto which only water was sprayed.
A 1 g lock of keratin fibres (90% Natural Grey NG hair) is wrapped around a brush (approximate diameter of 2 cm over a length of 3 cm).
2 g of an aqueous solution containing 10% am of polymer from Example 3 was prepared. This solution was vaporized (sprayed) onto the lock of keratin fibres.
The locks were weighed before and after application (0.5 g of water or of aqueous solution containing 10% am of polymer from Example 3 were deposited per lock).
Each lock was measured after application and 24 h after application (lock left at room temperature, 25° C.).

TABLE 10

| | Value after | Value after |
|---|---|---|
| Examples | application (in cm) | 24 h (in cm) |
| Untreated lock | 20.5 | 19 |
| Locke treated with water | 20.5 | 9 |
| Lock treated with an aqueous | 20.5 | 5 |
| solution containing 10% am | | |
| of polymer from Example 3 | | |

It appears that the locks which were treated with the composition according to the process of the invention make it possible to obtain curls which are, after 24 h, more wavy since the top of the curls is significantly closer according to the invention (5 cm) after 24 h vs. 9 cm with water alone and 19 cm without treatment.

1. Method for treating keratin materials, preferably α) keratin fibres, notably human keratin fibres such as the hair, or β) human skin, in particular the lips, which uses at least one composition, preferably a cosmetic composition, comprising a) one or more PHA copolymers a) comprising at least two different repeating polymer units chosen from the following units (A) and (B), and also the optical or geometric isomers thereof, the organic or mineral acid or base salts thereof, and the solvates thereof such as hydrates: 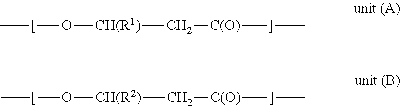
in which polymer units (A) and (B):
R¹ represents a saturated or unsaturated, linear or branched, non-cyclic hydrocarbon-based chain, or a saturated or unsaturated non-aromatic cyclic hydrocarbon-based chain, comprising from 3 to 30 carbon atoms; preferably, the hydrocarbon-based chain is chosen from i) linear or branched (C₅-C₂₈)alkyl, ii) linear or branched (C₆-C₂₈)alkenyl, iii) linear or branched (C₆-C₂₈)alkynyl, iii) preferably the hydrocarbon-based chain is non-cyclic and linear;
said hydrocarbon-based chain being:
substituted with one or more groups chosen from anionic groups (A⁻), cationic groups (C⁺) and mixtures thereof,
the A⁻ group(s) being chosen from —C(O)OH, —C(O)O⁻, —SO3H, —S(O)₂O—, —O—S(O)₂OH, —O—S(O)₂O⁻, —P(O)OH₂, —P(O)O₂⁻, —P(O)₂O⁻, —P(OH)₂⁻, ═P(O)—OH, —P(OH)O⁻, ═P(O)—O⁻, ═P—OH, and ═P—O⁻ and mixtures thereof, the anionic parts comprise one or more cationic counterions M⁺ to achieve the electroneutrality of the PHA;
the C⁺ cationic group(s) being chosen from cationizable amine groups —NR_{b}R_{c}, ammonium groups —N⁺RₐR_{b}R_{c}, phosphonium groups —P⁺RₐR_{b}R_{c}, cationizable amidine groups —C(═NRₐ)—NR_{b}R_{c}, amidinium groups —C(═NRₐ)—N⁺R_{b}R_{c}R_{d}, cationizable guanidine groups —N(Rₐ)—C(═NRₐ)—NR_{b}R_{c}, guanidinium groups —N(Rₐ)—C(═NRₐ)—N+R_{b}R_{c}R_{d}, or cationizable imidazolyl groups optionally substituted with one or more Rₐ radicals as defined below, or imidazolium groups optionally substituted with one or more Rₐ radicals as defined below, and mixtures thereof, the cationic parts comprising one or more anionic counterions Q⁻ to achieve the electroneutrality of the PHA, with
Rₐ representing a hydrogen atom, a (C₁-C₄)alkyl group, or (hetero)aryl(C₁-C₄)alkyl group such as benzyl, preferably Rₐ represents a hydrogen atom or a (C₁-C₄)alkyl group; R_{b}, R_{c}, and R_{d}, which may be identical or different, represent a (C₁-C₄)alkyl group optionally substituted with one or more hydroxyl groups, a (hetero)aryl group, a (hetero)aryl(C₁-C₄)alkyl group such as benzyl, a (hetero)cycloalkyl group or a (hetero)cycloalkyl(C₁-C₄)alkyl group, or else R_{b} and R_{c}, or R_{c} and R_{d} represent, with the nitrogen or phosphorus atom which bears them, a saturated or unsaturated, aromatic or non-aromatic heterocycle; particularly a (C₁-C₄)alkyl optionally substituted with one or more hydroxyl groups; the cationic parts comprising one or more anionic counterions Q⁻ to ensure electroneutrality;
it being possible for the R¹ radicals to also be substituted with one or more atoms or groups chosen from: a) halogen such as chlorine or bromine, b) hydroxy, c) thiol, d) (di)(C₁-C₄)(alkyl)amino, e) thiocarboxy, f) (thio)carboxamide —C(O)—N(Rₐ)₂ or —C(S)—N(Rₐ)₂, g) cyano, h) iso(thio)cyanate, i) (hetero)aryl such as phenyl or furyl, and j) (hetero)cycloalkyl, k) cosmetic active agent; l) R—X with R representing a group chosen from α) cycloalkyl such as cyclohexyl, 3) heterocycloalkyl such as sugar, preferably monosaccharide such as glucose, γ) (hetero)aryl such as phenyl, m) thiosulfate; X representing a′) O, S, N(R′ₐ) or Si(R′_{b})(R′_{c}), b′) S(O)ᵣ, or (thio)carbonyl, c′) or combinations of a′) with b′) such as (thio)ester, (thio)amide, (thio)urea or sulfonamide; R′ₐ representing a hydrogen atom, or a (C₁-C₄)alkyl group, or aryl(C₁-C₄)alkyl group such as benzyl, preferably R′ₐ represents a hydrogen atom; R′_{b} and R′_{c}, which may be identical or different, represent a (C₁-C₄)alkyl or (C₁-C₄)alkoxy group, particularly only one substituent, preferably chosen from a) halogen, and j) such as epoxide and
optionally interrupted with one or more a′) heteroatoms such as O, S, N(Rₐ) and Si(R_{b})(R_{c}), b′) S(O)ᵣ, (thio)carbonyl, c′) or combinations of a′) with b′) such as (thio)ester, (thio)amide, (thio)urea, sulfonamide, preferably ester —O—C(O)— or —C(O)—O— with r being equal to 1 or 2, Rₐ being as defined previously; preferably, Rₐ represents a hydrogen atom, R_{b} and R_{c} being as defined previously;
R² represents a hydrocarbon-based chain as defined for R¹; preferably the hydrocarbon-based chain of the radical R² has a carbon number corresponding to the number of carbon atoms in the radical R¹ minus at least one carbon atom, preferably corresponding to the number of carbon atoms in the radical R¹ minus two carbon atoms;
b) optionally water;
it being understood that:
the R¹ radicals may comprise, in the same PHA, A⁻ and C⁺ groups in which case the counterions M⁺ and Q⁻ may be absent while ensuring the electroneutrality of the PHA
(A) is different from (B);
the R¹ and R² radicals may comprise, in the same PHA, A⁻ and C⁺ groups in which case the counterions M⁺ and Q⁻ may be absent while ensuring the electroneutrality of the PHA;
the composition comprises an amount of less than or equal to 2% by weight of surfactants, relative to the total weight of the composition, particularly less than or equal to 1% by weight of surfactants, more particularly less than 0.5% by weight of surfactants, relative to the total weight of the composition, preferentially the composition is free of surfactant.

2. Method according to claim 1, which uses a composition comprising a) one or more PHA copolymers a) which comprise the repeating units chosen from those of formula (I), and also the optical or geometric isomers thereof, the organic or mineral acid or base salts thereof, and the solvates thereof such as hydrates: 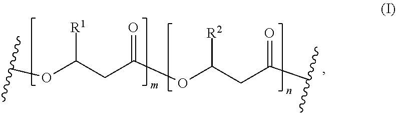
in which formula (I):
R¹ and R² are as defined in claim 1;
m and n are integers greater than or equal to 1; preferably, the sum n+m is inclusively between 450 and 1400; preferably m<n.

3. Method according to claim 1, using a composition comprising a) one or more PHA copolymers a) in which R¹ is a hydrocarbon-based chain which is 1) either substituted with one or more groups chosen from A⁻ and C⁺ as defined in claim 1, preferably substituted with one or more A⁻ groups, and is not interrupted, 2) or substituted with one or more groups chosen from A⁻ and C⁺ as defined in claim 1, preferably substituted with one or more A⁻ groups, and interrupted with one or more heteroatoms or a′) to c′) groups as defined for R¹, preferably heteroatoms such as O, or S, preferably S, more particularly 2).

4. Method according to claim 1, using a composition, comprising a) one or more PHA copolymer(s) a) in which R1 represents a hydrocarbon-based chain, notably i) linear or branched (C5-C28)alkyl, which is substituted with one or more (preferably one) A− groups chosen from —C(O)—OH, —C(O)O—, —SO3H, —S(O)2O—, —O—S(O)2OH, —O—S(O)2O—, —P(O)OH2, —P(O)O2-, —P(O)2O—, —P(OH)2, ═P(O)—OH, —P(OH)O—, ═P(O)—O—, ═P—OH, and ═P—O— and mixtures thereof, more preferentially chosen from —C(O)—OH, —C(O)O—, —SO3H, —S(O)2O—, —P(O)OH2, and —P(O)O2-, more preferentially carboxylate —C(O)O—, sulfonate —S(O)2O—, and phosphonate —P(O)O2-, the anionic parts comprising one or more cationic counterions M+ to achieve the electroneutrality of the PHA, such as an alkali metal, and alkaline-earth metal, ammonium: RaRbRcRdN+, or phosphonium: RaRbRcRdP+, preferably ammonium, with Ra, Rb, Rc and Rd as defined in claim 1, preferably Ra represents a hydrogen atom; Rb and Rc, which may be identical or different, preferably identical, represent a linear or branched, preferably branched (C1-C4)alkyl group such as isopropyl or isobutyl, more preferentially isopropyl; and Rd represents a linear or branched (C1-C4)alkyl group, such as ethyl, more particularly M+ represents a diisopropylethylammonium group: (i-Pr)2N+(H)Et.

5. Method according to claim 1, using a composition, comprising a) one or more PHA copolymer(s) a) in which R1 is substituted at the end of the chain on the opposite side from the carbon atom which bears said R1 radical, with one or more groups chosen from anionic groups (A−), and cationic groups (C+), preferably a single anionic or cationic group.

6. Method according to claim 1, using a composition comprising a) one or more PHA copolymers a) in which R1 represents a i) linear or branched (C3-C28)alkyl group which is substituted by one or more (preferably one) A− groups chosen from —C(O)—OH, —C(O)O— M+ with M+ as defined in claim 4, more preferentially A− is a carboxylate group —C(O)O— M+ with M+ as defined in claim 4; more particularly R1 represents a (C3-C28)alkyl group substituted with one or more, preferably just one group, at the chain end, said group(s) being chosen from —C(O)O— M+ with M+ as defined in claim 4 and interrupted with a sulfur atom, preferably in the beta position with respect to —C(O)O— M+.

7. Method according to claim 1, using a composition comprising a) one or more PHA copolymers a) in which R¹ has the following formula —(CH2)r-X-(ALK)u-G ALK represents a linear or branched (C1-C10)alkylene, r represents an integer between 6 and 11 inclusive; u is 0 or 1; and G represents an A− group.

8. Method according to claim 1, using a composition comprising a) one or more PHA copolymers a) in which R² is chosen from a linear or branched (C₃-C₂₀)alkyl, and linear or branched (C₃-C₂₀)alkenyl, in particular a linear hydrocarbon-based group, more particularly (C₃-C₂₀)alkyl or (C₃-C₂₀)alkenyl, preferably the hydrocarbon-based group has a carbon number corresponding to the number of carbon atoms in the radical R¹ minus at least one carbon atom, preferably corresponding to the number of carbon atoms in the radical R¹ minus two carbon atoms; preferentially R² is a (C₄-C₆)alkyl group such as n-pentyl or n-hexyl.

9. Method according to claim 1, using a composition comprising a) one or more PHA copolymers a) chosen from:

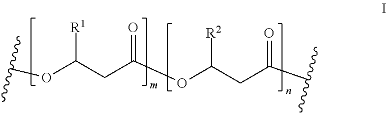

| Compound | R¹ | R² |
|---|---|---|
| (1A) | —(CH₂)ₚ—S—ALK₁—C(O)—OH | —AKL₂ |
| (2A) | —(CH₂)ₚ—S—ALK₁—(A⁻) | —AKL₂ |
| (3A) | —(CH₂)ₚ—S—ALK₁—(C⁺) | —AKL₂ |

p is an integer between 3 and 15,
m and n are integers,
ALK1 denotes a linear or branched divalent C1-C15 hydrocarbon-based radical,
ALK2 denotes a C3-C20 alkyl radical,
in particular, the stereochemistry of the carbon atoms bearing the R1 and R2 radicals is of the same (R) or (S) configuration, preferably of (R) configuration.

10. Method according to claim 1, wherein the PHA copolymer(s) a) are present in a content ranging from 0.1% to 65% by weight, relative to the total weight of the composition.
